(19) United States
(12) Patent Application Publication
(10) Pub. No.: US 2011/0130385 A1
(43) Pub. Date: Jun. 2, 2011

(54) Bicyclic Heterocylic Derivatives and Methods of Use

(76) Inventors: Manuel De Lera Ruiz, Branchburg, NJ (US); Michael Y. Berlin, Flemington, NJ (US); Junying Zheng, New Providence, NJ (US); Robert G. Aslanian, Rockaway, NJ (US); Kevin D. Mccormick, Basking Ridge, NJ (US)

(21) Appl. No.: 13/054,841

(22) PCT Filed: Jul. 21, 2009
(86) PCT No.: PCT/US09/51264

§ 371 Date: Feb. 15, 2011

Related U.S. Application Data
(60) Provisional application No. 61/082,949, filed on Jul. 23, 2008.

(51) Int. Cl. A61K 31/55; C07D 401/14; A61K 31/4725; A61P 11/00; A61P 9/00; A61P 29/00; A61P 1/00; A61P 3/00; A61P 25/00

(52) U.S. Cl. 514/217.01; 540/594; 546/148; 514/307

(57) ABSTRACT

The present invention relates to novel bicyclic heterocycle derivatives, pharmaceutical compositions comprising the bicyclic heterocycle derivatives and the use of these compounds for treating or preventing allergy, an allergy-induced airway response, congestion, a cardiovascular disease, an inflammatory disease, a gastrointestinal disorder, a neurological disorder, a metabolic disorder, obesity or an obesity-related disorder, diabetes, a diabetic complication, impaired glucose tolerance or impaired fasting glucose. R¹ is: I, Ia, Ib ou Ic. R² is alkyl, alkenyl, aryl, cycloalkyl. heterocycloalkyl or heteroaryl, any of which can be optionally substituted with R³; Y is —C(O)—, —S—, —S(O)—, —S(O)₂—, —CH₂— or —O—, such that if Y is —O— or —S—, then M is other than N and R¹ is (Ib);

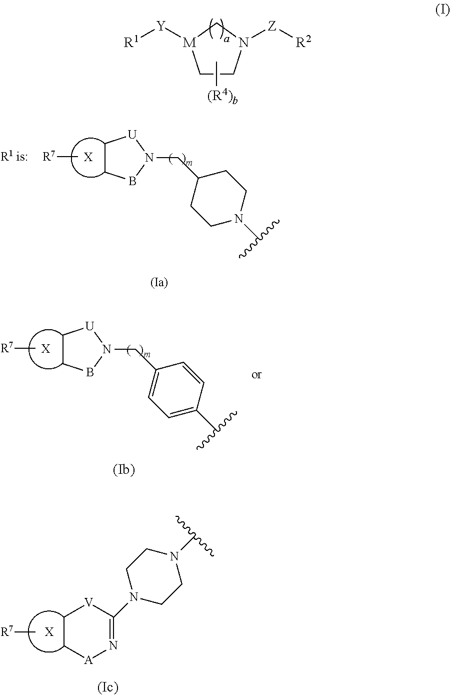

### FIELD OF THE INVENTION

The present invention relates to novel bicyclic heterocycle derivatives, pharmaceutical compositions comprising the bicyclic heterocycle derivatives and the use of these compounds for treating or preventing allergy, an allergy-induced airway response, congestion, a cardiovascular disease, an inflammatory disease, a gastrointestinal disorder, a neurological disorder, a metabolic disorder, obesity or an obesity-related disorder, diabetes, a diabetic complication, impaired glucose tolerance or impaired fasting glucose.

### BACKGROUND OF THE INVENTION

The histamine receptors, H₁, H₂ and H₃ are well-identified forms. The H₁ receptors are those that mediate the response antagonized by conventional antihistamines. H₁ receptors are present, for example, in the ileum, the skin, and the bronchial smooth muscle of humans and other mammals. Through H₂ receptor-mediated responses, histamine stimulates gastric acid secretion in mammals and the chronotropic effect in isolated mammalian atria.

H₃ receptor sites are found on sympathetic nerves, where they modulate sympathetic neurotransmission and attenuate a variety of end organ responses under control of the sympathetic nervous system. Specifically, H₃ receptor activation by histamine attenuates norepinephrine outflow to resistance and capacitance vessels, causing vasodilation.

Imidazole H₃ receptor antagonists are well known in the art. More recently, non-imidazole H₃ receptor antagonists have been disclosed in U.S. Pat. Nos. 6,720,328 and 6,849,621.

U.S. Pat. No. 5,869,479 discloses compositions for the treatment of the symptoms of allergic rhinitis using a combination of at least one histamine H₃ receptor antagonist and at least one histamine H₃ receptor antagonist.

Diabetes refers to a disease process derived from multiple causative factors and is characterized by elevated levels of plasma glucose, or hyperglycemia in the fasting state or after administration of glucose during an oral glucose tolerance test. Persistent or uncontrolled hyperglycemia is associated with increased and premature morbidity and mortality. Abnormal glucose homeostasis is associated with alterations of the lipid, lipoprotein and apolipoprotein metabolism and other metabolic and hemodynamic disease. As such, the diabetic patient is at especially increased risk of macrovascular and microvascular complications, including coronary heart disease, stroke, peripheral vascular disease, hypertension, nephropathy, neuropathy, and retinopathy. Accordingly, therapeutic control of glucose homeostasis, lipid metabolism and hypertension are critically important in the clinical management and treatment of diabetes mellitus.

There are two generally recognized forms of diabetes. In type I diabetes, or insulin-dependent diabetes mellitus (IDDM), patients produce little or no insulin, the hormone which regulates glucose utilization. In type 2 diabetes, or noninsulin dependent diabetes mellitus (NIDDM), patients often have plasma insulin levels that are the same or even elevated compared to nondiabetic subjects; however, these patients have developed a resistance to the insulin stimulating effect on glucose and lipid metabolism in the main insulin-sensitive tissue (muscle, liver and adipose tissue), and the plasma insulin levels, while elevated, are insufficient to overcome the pronounced insulin resistance.

Insulin resistance is not associated with a diminished number of insulin receptors but rather to a post-insulin receptor binding defect that is not well understood. This resistance to insulin responsiveness results in insufficient insulin activation of glucose uptake, oxidation and storage in muscle, and inadequate insulin repression of lipolysis in adipose tissue and of glucose production and secretion in the liver.

The available treatments for type 2 diabetes, which have not changed substantially in many years, have recognized limitations. While physical exercise and reductions in dietary intake of calories will dramatically improve the diabetic condition, compliance with this treatment is very poor because of well-entrenched sedentary lifestyles and excess food consumption, especially of foods containing high amounts of saturated fat. Increasing the plasma level of insulin by administration of sulfonylureas (e.g., tolbutamide and glipizide) or meglitinide, which stimulate the pancreatic [beta]-cells to secrete more insulin, and/or by injection of insulin when sulfonylureas or meglitinide become ineffective, can result in insulin concentrations high enough to stimulate the very insulin-resistant tissues. However, dangerously low levels of plasma glucose can result from administration of insulin or insulin secretagogues (sulfonylureas or meglitinide), and an increased level of insulin resistance due to the even higher plasma insulin levels can occur. The biguanides are a class of agents that can increase insulin sensitivity and bring about some degree of correction of hyperglycemia. However, the biguanides can induce lactic acidosis and nausea/diarrhea.

The glitazones 5-benzylthiazolidine-2,4-diones) are a separate class of compounds with potential for the treatment of type 2 diabetes. These agents increase insulin sensitivity in muscle, liver and adipose tissue in several animal models of type 2 diabetes, resulting in partial or complete correction of the elevated plasma levels of glucose without occurrence of hypoglycemia. The glitazones that are currently marketed are agonists of the peroxisome proliferator activated receptor (PPAR), primarily the PPAR-gamma subtype. PPAR-gamma agonism is generally believed to be responsible for the improved insulin sensititization that is observed with the glitazones. Newer PPAR agonists that are being tested for treatment of Type 2 diabetes are agonists of the alpha, gamma or delta subtype, or a combination of these, and in many cases are chemically different from the glitazones (i.e., they are not thiazolidinediones). Serious side effects (e.g., liver toxicity) have been noted in some patients treated with glitazone drugs, such as troglitazone.

Additional methods of treating the disease are currently under investigation. New biochemical approaches include treatment with alpha-glucosidase inhibitors (e.g., acarbose) and protein tyrosine phosphatase-1B (PTP-1B) inhibitors.

Compounds that are inhibitors of the dipeptidyl peptidase-IV (DPP-IV) enzyme are also under investigation as drugs that may be useful in the treatment of diabetes, and particularly type 2 diabetes.

Despite a widening body of knowledge concerning the treatment of diabetes, there remains a need in the art for small-molecule drugs with increased safety profiles and/or improved efficacy that are useful for the treatment of diabetes and related metabolic diseases. The present invention addresses that need.

### SUMMARY OF THE INVENTION

In one aspect, the present invention provides bicyclic heterocycle derivatives of Formula (I):

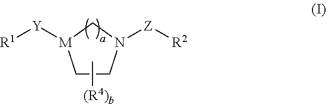

and pharmaceutically acceptable salts, solvates, esters and prodrugs thereof, wherein:

R¹ is:

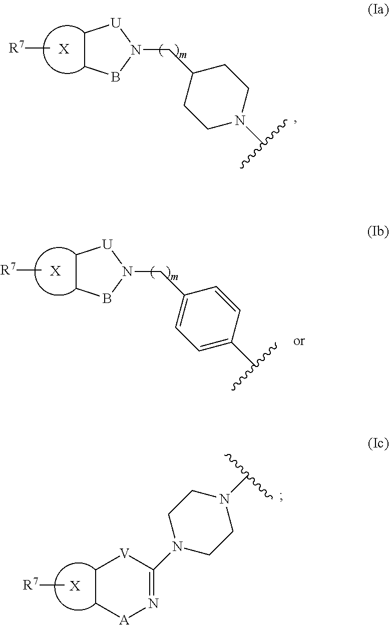

R² is alkyl, alkenyl, aryl, cycloalkyl, heterocycloalkyl or heteroaryl, any of which can be optionally substituted with R³;

R³ represents from 1 to 3 substituents, each independently selected from H, halo, alkyl, —OH, —O-alkyl, hydroxyalkyl, aryl, —O-aryl, haloalkyl, —NO₂, —C(O)OR⁹, —N(R⁹)₂, —C(O)N(R⁹)₂, -alkylene-N(R⁹)₂, —NHC(O)R⁹, —NHC(O)OR⁹, —NHS(O)₂R⁹, —S(O)₂N(R⁹)₂ and —CN;

R⁴ is halo, alkyl, —OH, —O-alkyl, haloalkyl or —CN;

each occurrence of R⁵ is independently H, alkyl, haloalkyl, aryl, heteroaryl, cycloalkyl or heterocycloalkyl;

R⁶ is alkyl, aryl or heteroaryl;

each occurrence of R⁷ is independently hydrogen, halo, —OH, alkyl, —O-alkyl, haloalkyl, —O-haloalkyl, —NO₂, —C(O)R⁵, —N(R⁵)₂, —C(O)N(R⁵)₂, —NHC(O)R⁵)₂, —NHS(O)₂R⁵, S(O)₂N(R⁵)₂ or —CN;

each occurrence of R⁸ is independently H or alkyl;

each occurrence of R⁹ is independently H, alkyl, haloalkyl, aryl, heteroaryl cycloalkyl or heterocycloalkyl;

each occurrence of R¹⁰ is independently hydrogen, halogen, —OH, alkyl, —O-alkyl, haloalkyl, —NO₂, —C(O)OR⁶, —N(C(O)N(R)₂, —NHC(O)R⁶, —NHS(O)₂R⁶, —S(O)₂N(R⁵)₂, —C(O)R⁵ or —CN;

R¹¹ is hydrogen, alkyl, —C(O)R⁶ or —S(O)₂R⁶;

R¹² is hydrogen, alkyl, haloalkyl or —C(O)R⁵;

R¹³ is hydrogen, alkyl or haloalkyl;

R¹⁴ represents 1 to 3 substituents, each independently selected from the group consisting of H, cycloalkyl, heterocycloalkyl, aryl, heteroaryl, haloalkyl, halo, —CN, —OH, —O-alkyl, —O-haloalkyl, —NO₂, and —N(R⁸)₂;

R¹⁵ represents 1 to 3 substituents, each independently selected from cycloalkyl, heterocycloalkyl, aryl, heteroaryl and haloalkyl;

A is a bond, —O—, —S—, —O-alkylene-, —S-alkylene-, —C(R¹⁰)ₚC(R¹²) or —N(R¹²⁾C(R¹⁰)ₚC(R¹²)—;

B is a bond, —O—, —S—, —O-alkylene-, —S-alkylene-, —C(R¹⁰)ₚC(R¹²)— or —N(R¹²)—C(R¹⁰)ₚC(R¹²)—, such that when R¹ is (Ia) or (Ib) and B is —O— or —S—, then U is other than —O— or —S—;

M is —CH—, —C(halo)- or —N—;

Q is —O—, —S—, —S(O)— or —S(O)₂—;

U is a bond, —O—, —S—, —O-alkylene-, —S-alkylene-, —C(R¹⁰)ₙC(R¹²)—, —N(R¹¹)C(R¹²)C(R¹²)—, —N(R¹¹)C(R¹²)_{c}(R¹⁰)C(R¹²)—, -QC(R¹³)C(R¹⁰)C(R¹²)—, —C(R¹²)N(R¹¹)C(R¹²)C(R¹²)—, -QC(R¹³)C(R¹²)—, or —C(R¹³)QC(R¹³)C(R¹²)—, such that when R¹ is (Ia) or (Ib) and B is —O— or —S—, then U is other than —O— or —S—;

V is a bond, —O—, —S—, —O-alkylene-, —S-alkylene-, —C(R¹⁰)ₚC(R¹²)—, C(R¹²)N(R¹¹)C(R¹²)—, —C(R¹³)QC(R¹³)—, —N(R¹¹)C(R¹²)C(R¹⁰)—, -QC(R¹³)C(R¹⁰)—, —N(R¹¹)C(R¹²)— or -QC(R¹³)—;

X is aryl, or heteroaryl, each of which can be optionally fused to a benzene ring;

Y is —C(O)—, —S—, —S(O)—, —S(O)₂—, —CH₂— or —O—, such that if Y is —O— or —S—, then M is other than N and R¹ is (Ib);

Z is a bond, alkylene, -alkylene-cycloalkylene-alkylene-, -alkylene-heterocycloalkylene-alkylene-, —CH(R⁸)—CH(R⁸)—O—, —CH(R⁸)—CH(R⁸)—N—, —CH(R⁸)—(C₁-C₅ alkylene)-R¹⁴, —CH(R⁸)═C(R⁸)—, —C(R⁸)—, —CH(R⁸)—C(R⁸)═C(R⁸)—(C₁-C₃ alkylene)-R¹⁴, wherein any alkylene moiety of a Z group can be optionally substituted with one or more R¹⁵ groups, such that when Z is an -alkylene-heterocycloalkylene-alkylene-group substituted with one or more R¹⁵ groups and the heterocycloalkylene group is bonded through a ring nitrogen atom, then Z is —(C₂-C₄ alkylene)-heterocycloalkylene-alkylene-;

a is 1, 2, or 3;

b is 0.1, or 2;

m is 0, 1 or 2; and

each occurrence of p is 0, 1, 2 or 3.

The Compounds of Formula (I) and pharmaceutically acceptable salts, solvates, prodrugs and esters thereof can be useful for treating or preventing allergy, an allergy-induced airway response, congestion, a cardiovascular disease, an inflammatory disease, a gastrointestinal disorder, a neurological disorder, a metabolic disorder, obesity or an obesity-related disorder, diabetes, a diabetic complication, impaired glucose tolerance or impaired fasting glucose (each being a “Condition”) in a patient.

Also provided by the invention are methods for treating or preventing Condition in a patient, comprising administering to the patient an effective amount of one or more compounds of Formula (I).

In addition, the present invention provides methods for treating or preventing Condition in a patient, comprising administering to the patient one or more Compounds of Formula (I) and an additional therapeutic agent that is not a Compound of Formula (I), wherein the amounts administered are together effective to treat or prevent the Condition.

The present invention further provides pharmaceutical compositions comprising an effective amount of one or more compounds of Formula (I) or a pharmaceutically acceptable salt, solvate thereof, and a pharmaceutically acceptable carrier. The compositions can be useful for treating or preventing a Condition in a patient.

The details of the invention are set forth in the accompanying detailed description below.

Although any methods and materials similar to those described herein can be used in the practice or testing of the present invention, illustrative methods and materials are now described. Other features, objects, and advantages of the invention will be apparent from the description and the claims. All patents and publications cited in this specification are incorporated herein by reference.

### DETAILED DESCRIPTION OF THE INVENTION

The term “patient” as used herein, refers to a human or non-human mammal. In one embodiment, a patient is a human. In another embodiment, a patient is a non-human mammal, including, but not limited to, a monkey, dog, baboon, rhesus, mouse, rat, horse, cat or rabbit. In another embodiment, a patient is a companion animal, including but not limited to a dog, cat, rabbit, horse or ferret. In one embodiment, a patient is a dog. In another embodiment, a patient is a cat.

The term “obesity” as used herein, refers to a patient being overweight and having a body mass index (BMI) of 25 or greater. In one embodiment, an obese patient has a BMI of about 25 or greater. In another embodiment, an obese patient has a BMI of between about 25 and about 30. In another embodiment, an obese patient has a BMI of between about 35 and about 40. In still another embodiment, an obese patient has a BMI greater than 40.

The term “obesity-related disorder” as used herein refers to: (i) disorders which result from a patient having a BMI of about 25 or greater; and (ii) eating disorders and other disorders associated with excessive food intake. Non-limiting examples of an obesity-related disorder include edema, shortness of breath, sleep apnea, skin disorders and high blood pressure.

The term “metabolic syndrome” as used herein, refers to a set of risk factors that make a patient more succeptible to cardiovascular disease and/or type 2 diabetes. As defined herein, a patient is considered to have metabolic syndrome if the patient has one or more of the following five risk factors:
1) central/abdominal obesity as measured by a waist circumference of greater than 40 inches in a male and greater than 35 inches in a female;
2) a fasting triglyceride level of greater than or equal to 150 mg/dL;
3) an HDL cholesterol level in a male of less than 40 mg/dL or in a female of less than 50 mg/dL;
4) blood pressure greater than or equal to 130/85 mm Hg; and
5) a fasting glucose level of greater than or equal to 110 mg/dL.

The term “impaired glucose tolerance” as used herein, is defined as a two-hour glucose level of 140 to 199 mg per dL (7.8 to 11.0 mmol) as measured using the 75-g oral glucose tolerance test. A patient is said to be under the condition of impaired glucose tolerance when he/she has an intermediately raised glucose level after 2 hours, wherein the level is less than would qualify for type 2 diabetes mellitus.

The term “impaired fasting glucose” as used herein, is defined as a fasting plasma glucose level of 100 to 125 mg/dL; normal fasting glucose values are below 100 mg per dL.

The term “upper airway” as used herein, refers to the upper respiratory system, i.e., the nose, throat, and associated structures.

The term “effective amount” as used herein, refers to an amount of compound of formula (I) and/or an additional therapeutic agent, or a composition thereof that is effective in producing the desired therapeutic, ameliorative, inhibitory or preventative effect when administered to a patient suffering from a Condition. In the combination therapies of the present invention, an effective amount can refer to each individual agent or to the combination as a whole, wherein the amounts of all agents administered are together effective, but wherein the component agent of the combination may not be present individually in an effective amount.

The term “alkyl,” as used herein, refers to an aliphatic hydrocarbon group which may be straight or branched and which contains from about 1 to about 20 carbon atoms. In one embodiment, an alkyl group contains from about 1 to about 12 carbon atoms. In another embodiment, an alkyl group contains from about 1 to about 6 carbon atoms. Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl and neohexyl. An alkyl group may be unsubstituted or substituted by one or more substituents which may be the same or different, each substituent being independently selected from the group consisting of halo, alkyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. In one embodiment, an alkyl group is unsubstituted. In another embodiment, an alkyl group is linear. In another embodiment, an alkyl group is branched.

The term “alkenyl,” as used herein, refers to an aliphatic hydrocarbon group containing at least one carbon-carbon double bond and which may be straight or branched and contains from about 2 to about 15 carbon atoms. In one embodiment, an alkenyl group contains from about 2 to about 12 carbon atoms. In another embodiment, an alkenyl group contains from about 2 to about 6 carbon atoms. Non-limiting examples of alkenyl groups include ethenyl, propenyl, n-butenyl, 3-methylbut-2-enyl, n-pentenyl, octenyl and decenyl. An alkenyl group may be unsubstituted or substituted by one or more substituents which may be the same or different, each substituent being independently selected from the group consisting of halo, alkyl, aryl, cycloalkyl, cyano, —O-alkyl and —S(alkyl). In one embodiment, an alkenyl group is unsubstituted.

The term “alkynyl,” as used herein, refers to an aliphatic hydrocarbon group containing at least one carbon-carbon triple bond and which may be straight or branched and contains from about 2 to about 15 carbon atoms. In one embodiment, an alkynyl group contains from about 2 to about 12 carbon atoms. In another embodiment, an alkynyl group contains from about 2 to about 6 carbon atoms. Non-limiting examples of alkynyl groups include ethynyl, propynyl, 2-butynyl and 3-methylbutynyl. An alkynyl group may be unsubstituted or substituted by one or more substituents which may be the same or different, each substituent being independently selected from the group consisting of alkyl, aryl and cycloalkyl. In one embodiment, an alkynyl group is unsubstituted.

The term “alkylene,” as used herein, refers to an alkyl group, as defined above, wherein one of the alkyl group's hydrogen atoms has been replaced with a bond. Non-limiting examples of alkylene groups include —CH₂—, —CH₂CH₂—, —CH₂CH₂CH₂—, —CH₂CH₂CH₂CH₂—, —CH(CH₃)CH₂CH₂— and —CH₂CH(CH₃)CH₂—. An alkylene group may be unsubstituted or substituted by one or more substituents which may be the same or different, each substituent being independently selected from the group consisting of halo, alkyl, aryl, cycloalkyl, cyano, —O-alkyl and —S(alkyl). In one embodiment, an alkylene group is unsubstituted. In another embodiment, an alkylene group has from 1 to about 6 carbon atoms. In another embodiment, an alkylene group is branched. In still another embodiment, an alkylene group is linear.

The term “alkenylene,” as used herein, refers to an alkenyl group, as defined above, wherein one of the alkenyl group's hydrogen atoms has been replaced with a bond. Non-limiting examples of alkenylene groups include —CH═CH—, —CH₂CH═CH—, —CH₂CH═CHCH₂—, —CH═CHCH₂CH₂—, —CH₂CHCH═CH—, —CH(CH₃)CH═CH— and —CH═C(CH₃)CH₂—. In one embodiment, an alkenylene group has from 2 to about 6 carbon atoms. In another embodiment, an alkenylene group is branched. In another embodiment, an alkenylene group is linear.

The term “alkynylene,” as used herein, refers to an alkynyl group, as defined above, wherein one of the alkynyl group's hydrogen atoms has been replaced with a bond. Non-limiting examples of alkynylene groups include —C≡C—, —CH₂C≡C—, —CH₂C≡CCH₂—, —C≡CCH₂CH₂—, —CH₂CHC≡C—, —CH(CH₃)C≡C— and —C≡CCH₂—. In one embodiment, an alkynylene group has from 2 to about 6 carbon atoms. In another embodiment, an alkynylene group is branched. In another embodiment, an alkynylene group is linear.

The term “aryl” as used herein, refers to an aromatic monocyclic or multicyclic ring system comprising from about 6 to about 14 carbon atoms. In one embodiment, an aryl group contains from about 6 to about 10 carbon atoms. An aryl group can be optionally substituted with one or more “ring system substituents” which may be the same or different, and are as defined herein below. Non-limiting examples of aryl groups include phenyl and naphthyl. In one embodiment, an aryl group is unsubstituted. In another embodiment, an aryl group is phenyl.

The term “cycloalkyl,” as used herein, refers to a non-aromatic mono- or multicyclic ring system comprising from about 3 to about 10 ring carbon atoms. In one embodiment, a cycloalkyl contains from about 3 to about 7 ring carbon atoms. In another embodiment, a cycloalkyl contains from about 5 to about 7 ring atoms. Non-limiting examples of monocyclic cycloalkyls include cyclopropyl, cyclobutyl, cyclopentyl, cyclohexyl, cycloheptyl and cyclooctyl. Non-limiting examples of multicyclic cycloalkyls include 1-decalinyl, norbornyl and adamantyl. A cycloalkyl group can be optionally substituted with one or more “ring system substituents” which may be the same or different, and are as defined herein below. In one embodiment, a cycloalkyl group is unsubstituted.

The term “cycloalkenyl,” as used herein, refers to a non-aromatic mono- or multicyclic ring system comprising from about 3 to about 10 ring carbon atoms and containing at least one endocyclic double bond. In one embodiment, a cycloalkenyl contains from about 5 to about 10 ring carbon atoms. In another embodiment, a cycloalkenyl contains 5 or 6 ring atoms. Non-limiting examples of monocyclic cycloalkenyls include cyclopentenyl, cyclohexenyl, cyclohepta-1,3-dienyl, and the like. A cycloalkenyl group can be optionally substituted with one or more “ring system substituents” which may be the same or different, and are as defined herein below. In one embodiment, a cycloalkenyl group is unsubstituted. In another embodiment, a cycloalkenyl group is a 6-membered cycloalkenyl. In another embodiment, a cycloalkenyl group is a 5-membered cycloalkenyl.

The term “heteroaryl,” as used herein, refers to an aromatic monocyclic or multicyclic ring system comprising about 5 to about 14 ring atoms, wherein from 1 to 4 of the ring atoms is independently O, N or S and the remaining ring atoms are carbon atoms. In one embodiment, a heteroaryl group has 5 to 10 ring atoms. In another embodiment, a heteroaryl group is monocyclic and has 5 or 6 ring atoms. A heteroaryl group can be optionally substituted by one or more “ring system substituents” which may be the same or different, and are as defined herein below. A heteroaryl group is attached via a ring carbon atom, and any nitrogen atom of a heteroaryl can be optionally oxidized to the corresponding N-oxide. The term “heteroaryl” also encompasses a heteroaryl group, as defined above, which has been fused to a benzene ring. Non-limiting examples of heteroaryls include pyridyl, pyrazinyl, furanyl, thienyl, pyrimidinyl, isoxazolyl, isothiazolyl, oxazolyl, thiazolyl, pyrazolyl, furazanyl, pyrrolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. In one embodiment, a heteroaryl group is unsubstituted. In another embodiment, a heteroaryl group is a 6-membered heteroaryl. In another embodiment, a heteroaryl group is a 5-membered heteroaryl.

The term “heterocycloalkyl,” as used herein, refers to a non-aromatic saturated monocyclic or multicyclic ring system comprising 3 to about 10 ring atoms, wherein from 1 to 4 of the ring atoms are independently O, S or N and the remainder of the ring atoms are carbon atoms. In one embodiment, a heterocycloalkyl group has from about 5 to about 10 ring atoms. In another embodiment, a heterocycloalkyl group has 5 or 6 ring atoms. There are no adjacent oxygen and/or sulfur atoms present in the ring system. Any —NH group in a heterocycloalkyl ring may exist protected such as, for example, as an —N(BOC), —N(Cbz), —N(Tos) group and the like; such protected heterocycloalkyl groups are considered part of this invention. A heterocycloalkyl group can be optionally substituted by one or more “ring system substituents” which may be the same or different, and are as defined herein below. The nitrogen or sulfur atom of the heterocycloalkyl can be optionally oxidized to the corresponding N-oxide, S-oxide or S,S-dioxide. Non-limiting examples of monocyclic heterocycloalkyl rings include piperidyl, pyrrolidinyl, piperazinyl, pyrrolidonyl, morpholinyl, thiomorpholinyl, thiazolidinyl, 1,4-dioxanyl, tetrahydrofuranyl, tetrahydrothiophenyl, lactam, lactone, and the like. A ring carbon atom of a heterocycloalkyl group may be functionalized as a carbonyl group. An illustrative example of such a heterocycloalkyl group is pyrrolidonyl:

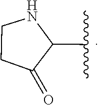

In one embodiment, a heterocycloalkyl group is unsubstituted. In another embodiment, a heterocycloalkyl group is a 6-membered heterocycloalkyl. In another embodiment, a heterocycloalkyl group is a 5-membered heterocycloalkyl.

The term “heterocycloalkenyl,” as used herein, refers to a heterocycloalkyl group, as defined above, wherein the heterocycloalkyl group contains from 3 to 10 ring atoms, and at least one endocyclic carbon-carbon or carbon-nitrogen double bond. In one embodiment, a heterocycloalkenyl group has from 5 to 10 ring atoms. In another embodiment, a heterocycloalkenyl group is monocyclic and has 5 or 6 ring atoms. A heterocycloalkenyl group can be optionally substituted by one or more ring system substituents, wherein “ring system substituent” is as defined above. The nitrogen or sulfur atom of the heterocycloalkenyl can be optionally oxidized to the corresponding N-oxide, S-oxide or S,S-dioxide. Non-limiting examples of heterocycloalkenyl groups include tetrahydroisoquinolyl, tetrahydroquinolyl 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluoro-substituted dihydrofuranyl, 7-oxabicyclo[2.2.1]heptenyl, dihydrothiophenyl, dihydrothiopyranyl, and the like. A ring carbon atom of a heterocycloalkenyl group may be functionalized as a carbonyl group, for example:

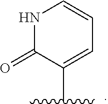

In one embodiment, a heterocycloalkenyl group is unsubstituted. In another embodiment, a heterocycloalkenyl group is a 6-membered heterocycloalkenyl. In another embodiment, a heterocycloalkenyl group is a 5-membered heterocycloalkenyl.

It should also be noted that tautomeric forms such as, for example, the moieties:

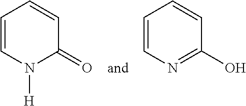

are considered equivalent in certain embodiments of this invention.

The term “ring system substituent,” as used herein, refers to a substituent group attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, -alkylene-aryl, -alkylene-heteroaryl, -alkenylene-heteroaryl, -alkynylene-heteroaryl, hydroxy, hydroxyalkyl, haloalkyl, —O-alkyl, -alkylene-O-alkyl, —O-aryl, ar-O-alkyl, acyl, aroyl, halo, nitro, cyano, carboxy, —C(O)O-alkyl, —C(O)O-aryl, —C(O)O-alkelene-aryl, —S(O)-alkyl, —S(O)₂-alkyl, —S(O)-aryl, —S(O)₂-aryl, —S(O)-heteroaryl, —S(O)₂-heteroaryl, —S-alkyl, —S-aryl, —S-heteroaryl, —S-alkylene-aryl, —S-alkylene-heteroaryl, cycloalkyl, heterocycloalkyl, —O—C(O)-alkyl, —O—C(O)-aryl, —O—C(O)-cycloalkyl, —C(═NH)—NH₂, —C(—NH)—NH(alkyl), Y₁Y₂N—, Y₁Y₂N-alkyl-, Y₁Y₂NC(O)— and Y₁Y₂NSO₂—, wherein Y₁ and Y₂ can be the same or different and are independently selected from the group consisting of H, alkyl, aryl, cycloalkyl, and -alkylene-aryl. “Ring system substituent” may also mean a single moiety which simultaneously replaces two available hydrogens on two adjacent carbon atoms (one H on each carbon) on a ring system. Examples of such moiety are methylenedioxy, ethylenedioxy, —C(CH₃)₂— and the like which form moieties such as, for example:

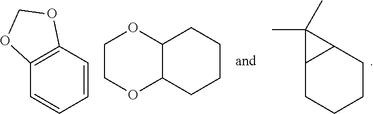

“Halo” means —F, —Cl, —Br or —I. In one embodiment, halo refers to —Cl or —Br.

The term “haloalkyl,” as used herein, refers to an alkyl group as defined above, wherein one or more of the alkyl group's hydrogen atoms has been replaced with a halogen. In one embodiment, a haloalkyl group has from 1 to 6 carbon atoms. In another embodiment, a haloalkyl group is substituted with from Ito 3 F atoms. Non-limiting examples of haloalkyl groups include —CH₂F, —CHF₂, —CH₂Cl and —CCl₃.

The term “hydroxyalkyl,” as used herein, refers to an alkyl group as defined above, wherein one or more of the alkyl group's hydrogen atoms has been replaced with an —OH group. In one embodiment, a hydroxyalkyl group has from 1 to 6 carbon atoms. Ion limiting examples of hydroxyalkyl groups include —CH₂OH, —CH₂CH₂OH, —CH₂CH₂CH₂OH and —CH₂CH(OH)CH₃.

The term “alkoxy” as used herein, refers to an —O-alkyl group, wherein an alkyl group is as defined above. Non-limiting examples of —O-alkyl groups include methoxy, ethoxy, n-propoxy, isopropoxy, n-butoxy and t-butoxy. An —O-alkyl group is bonded via its oxygen atom.

The term “substituted” means that one or more hydrogens on the designated atom is replaced with a selection from the indicated group, such that the designated atom's normal valency under the existing circumstances is not exceeded, and that the substitution results in a stable compound. Combinations of substituents and/or variables are permissible only if such combinations result in stable compounds. By “stable compound’ or “stable structure” is meant a compound that is sufficiently robust to survive isolation to a useful degree of purity from a reaction mixture, and formulation into an efficacious therapeutic agent.

The term “purified”, “in purified form” or “in isolated and purified form” for a compound refers to the physical state of the compound after being isolated from a synthetic process (e.g., from a reaction mixture), or natural source or combination thereof. Thus, the term “purified”, “in purified form” or “in isolated and purified form” for a compound refers to the physical state of the compound after being obtained from a purification process or processes described herein or well known to the skilled artisan (e.g., chromatography, recrystallization and the like), in sufficient purity to be characterizable by standard analytical techniques described herein or well known to the skilled artisan.

It should also be noted that any carbon as well as heteroatom with unsatisfied valences in the text, schemes, examples and Tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in Organic Synthesis (1991), Wiley, New York.

When any variable (e.g., aryl, heterocycle, R², etc.) occurs more than one time in any constituent or in Formula (I), its definition on each occurrence is independent of its definition at every other occurrence, unless otherwise noted.

Prodrugs and solvates of the compounds of the invention are also contemplated herein. A discussion of prodrugs is provided in T. Higuchi and V. Stella, Pro-drugs as Novel Delivery Systems (1.987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g, a drug precursor) that is transformed in viva to yield a Compound of Formula (I) or a pharmaceutically acceptable salt, hydrate or solvate of the compound. The transformation may occur by various mechanisms (e.g. by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a Compound of Formula (I) or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, —O-alkylcarbonyloxymethyl having from 3 to 6 carbon atoms, 1-(—O-alkylcarbonyloxy)ethyl having from 4 to 7 carbon atoms, 1-methyl-1-(—O-alkylcarbonyloxy)ethyl having from 5 to 8 carbon atoms, N—(—O-alkylcarbonyl)aminomethyl having from 3 to 9 carbon atoms, 1-(N—(—O-alkylcarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as (3-dimethylaminoethyl), carbamoyl-(C₁-C₂)alkyl, N,N-di (C₁-C₂)alkylcarbamoyl-(C₁-C₂)alkyl and piperidino-, pyrrolidino- or morpholino(C₂-C₃)alkyl, and the like.

Similarly, if a Compound of Formula (I) contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₄)—O-alkylcarbonyloxymethyl, N—(C₁-C₆)—O-alkylcarbonylaminomethyl, succinoyl, (C₁-C₆)alkanoyl, α-amino(C₁-C₄)alkyl, α-amino(C₁-C₄)alkylene-aryl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a —OH group of the hemiacetal form of a carbohydrate), and the like.

If a Compound of Formula (I) incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇) cycloalkyl, benzyl, or R-carbonyl is a natural α-aminoacyl, —C(OH)C(O)OY¹ wherein Y¹ is H, (Cₜ—C₆)alkyl or benzyl, —C(OY²)Y³ wherein Y² is (Cᵣ—C₄) alkyl and Y³ is (C₁-C₆)alkyl, carboxy (C₁-C₆)alkyl, amino(C₁-C₄)alkyl or mono-N— or di-N,N—(C₁-C₆)alkylaminoalkyl, —C(Y⁴)Y⁵ wherein Y⁴ is H or methyl and Y³ is mono-N— or di-N,N—(C₁-C₆)alkylamino morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of solvates include ethanolates, methanolates, and the like. “Hydrate” is a solvate wherein the solvent molecule is H₂O.

One or more compounds of the invention may optionally be converted to a solvate. Preparation of Solvates is Generally Known. Thus, for Example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTechours., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

The Compounds of Formula (I) can form salts which are also within the scope of this invention. Reference to a Compound of Formula (I) herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a Compound of Formula (I) contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the Formula (I) may be formed, for example, by reacting a Compound of Formula (I) with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamine, choline, t butyl amine, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the hydroxy group of a —OH compound, in which the non-carbonyl moiety of the carboxylic acid portion of the ester grouping is selected from straight or branched chain alkyl (for example, methyl, ethyl, n-propyl, isopropyl, t-butyl, sec-butyl or n-butyl), —O-alkylalkyl (for example, methoxymethyl), aralkyl (for example, benzyl), —O-alkylene-aryl (for example, phenoxymethyl), aryl (for example, phenyl optionally substituted with, for example, halo, C₁₋₄alkyl, or C₁₋₄—O-alkyl or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

Diastereomeric mixtures can be separated into their individual diastereorners on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Sterochernically pure compounds may also be prepared by using chiral starting materials or by employing salt resolution techniques. Also, some of the Compounds of Formula (I) may be atropisomers (e.g., substituted biaryls) and are considered as part of this invention. Enantiomers can also be separated by use of chiral HPLC column.

It is also possible that the Compounds of Formula (I) may exist in different tautomeric ms, and all such forms are embraced within the scope of the invention. Also, for example, all keto-enol and imine-enamine forms of the compounds are included in the invention.

All stereoisomers (for example, geometric isomers, optical isomers and the like) of the present compounds (including those of the salts, solvates, hydrates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl), (For example, if a Compound of Formula (I) incorporates a double bond or a fused ring, both the cis- and trans-forms, as well as mixtures, are embraced within the scope of the invention. Also, for example, all keto-enol and imine-enamine forms of the compounds are included in the invention).

Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt” “solvate”, “ester”, “prodrug” and the like, is intended to apply equally to the salt, solvate, ester and prodrug of enantiomers, stereoisomers, rotamers, tautomers, positional isomers, racemates or prodrugs of the inventive compounds.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of H, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labelled Compounds of Formula (I) (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some instances. In one embodiment, one or more hydrogen atoms of a compound of formula (I) is replaced with a deuterium atom. Isotopically labelled Compounds of Formula (I) can generally be prepared using synthetic chemical procedures analogous to those disclosed herein for making the Compounds of Formula (I), by substituting an appropriate isotopically labelled starting material or reagent for a non-isotopically labelled starting material or reagent.

Polymorphic forms of the Compounds of Formula (I), and of the salts, solvates, hydrates, esters and prodrugs of the Compounds of Formula (I), are intended to be included in the present invention.

Unless otherwise stated, the following abbreviations have the stated meanings: boc or BOC is tert-butyoxycarbonyl, BtOH is butanol, tBuOH is tertiary-butanol, DCM is dichloromethane, DIPEA is diisopropylethylamine, DMAP is N,N′-dimethylaminopyridine, DMF is N,N-dimethylformamide, DMSO is dimethylsulfoxide, DPPA is diphenylphosphoryl azide, EDC is 1,2-dichloroethane, Et₃N is triethylamine, EtOAc is ethyl acetate, EtOH is ethanol, Et₃SiH is triethylsilyl hydride, HbAlC is glycosylated hemoglobin, HOBt is N-hydroxybenzotriazole, i-Pr is isopropyl, KHMDS is potassium hexamethyldisilazide, Me is methyl, Et is ethyl, MeOH is methanol, NaBH(OAc)₃ is sodium triacetoxyborohydride, NBS is N-bromosuccinimide, Ph is phenyl, PPh₃ is triphenylphosphine, Ra—Ni is Raney nickel, TFA is trifluoroacetic acid, THF is tetrahydrofuran and TLC is thin layer chromatography.

### The Compounds of Formula (I)

The present invention provides Compounds of Formula (I):

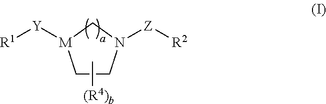

and pharmaceutically acceptable salts and solvates thereof, wherein R¹, R², R⁴, M, Y, Z, a and b are defined above for the Compounds of Formula (I).

In one embodiment, R¹ has the Formula (Ia).

In another embodiment, R¹ has the Formula (Ib).

In another embodiment, R¹ has the Formula (Ic).

In one embodiment, R¹ has the Formula (Ia) and R⁷ is selected from halo, —O-alkyl or —CN.

In another embodiment, R¹ has the Formula (Ib) and R⁷ is selected from halo, —O-alkyl or —CN.

In another embodiment, R¹ has the Formula (Ic) and R⁷ is selected from halo, —O-alkyl or —CN.

In one embodiment, R¹ is:

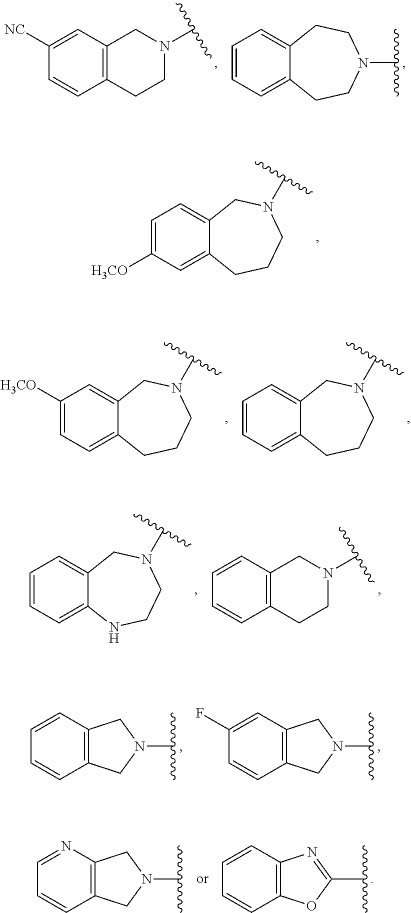

In one embodiment, R² is R³-heteroaryl.

In another embodiment, R² is R³-heteroaryl, wherein R³ is H, alkyl or —N(R₉)₂.

In another embodiment, R² is R³-heteroaryl, wherein R³ is H, methyl or —NH₂.

In still another embodiment, R² is R³-heteroaryl, wherein the heteroaryl is a 6-membered heteroaryl.

In another embodiment, R² is R³-heteroaryl, wherein the heteroaryl is a 5-membered heteroaryl.

In another embodiment, R² is pyridyl, thiazolyl, pyridazinyl or pyrimidinyl, which can be optionally substituted.

In one embodiment. R² is pyridyl.

In another embodiment, R² is pyridazinyi.

In another embodiment, R² is pyrimidinyl.

In still another embodiment, R² is thiazolyl.

In yet another embodiment, R² is —NH₂ substituted heteroaryl.

In another embodiment, R² is —NH₂ substituted pyridyl.

In a further embodiment, R² is —NH₂ substituted thiazolyl.

In another embodiment, R² is —NH₂ substituted pyrimidinyl.

In another embodiment, R² is —NH₂ substituted pyridazinyl.

In one embodiment, R² is

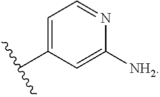

In one embodiment, m is 0.

In another embodiment, m is 1.

In one embodiment, U is —CH₂—.

In another embodiment, U is —CH₂—CH₂—.

In one embodiment, A is —CH₂—CH₂—.

In another embodiment, A is —CH₂—CH₂—CH₂—.

In another embodiment, A is —CH₂—CH₂—NH—, where the —NH— is attached to ring X.

In one embodiment, U is —CH₂— and A is —CH₂—CH₂—.

In another embodiment, U is and A are each —CH₂—CH₂—.

In another embodiment, U is —CH₂— and A is —CH₂—CH₂—CH₂—.

In one embodiment, ring X is optionally substituted phenyl.

In another embodiment, ring X is optionally substituted heteroaryl, which can be optionally fused to a benzene ring.

In another embodiment, ring X is an optionally substituted pyrrolyl ring.

In another embodiment, ring X is an optionally substituted benzo-fused pyrrolyl ring.

In one embodiment, Y is —C(O)—.

In another embodiment, Y is —O—.

In one embodiment, Y is —C(O)— and R¹ is (Ia).

In another embodiment, Y is —C(O)— and R¹ is (Ib).

In another embodiment, Y is —O— and R¹ is (Ic).

In one embodiment, Y is —C(O)—, is (Ia) and M is —CH— or —CF—.

In another embodiment, Y is —C(O)—, R¹ is (Ib) and M is —CH— or —C—

In another embodiment, Y is —O—, R¹ is (Ic) and M is —CH— or —CF—.

In one embodiment, Y is —C(O)—, R¹ is (Ia), Z is alkylene and R² is heteroaryl.

In another embodiment, Y is —C(O)—, R³ is (Ib), Z is alkylene and R² is heteroaryl.

In another embodiment. Y is —O—, R¹ is (Ic), Z is alkylene and R² is heteroaryl.

In one embodiment, a is 2, Y is —C(O)—, R¹ is (Ia), Z is alkylene and R² is heteroaryl.

In another embodiment, a is 2, Y is —C(O)—; R¹ is (Ib); Z is alkylene and R² is heteroaryl.

In another embodiment, a is 2; Y is —O—; R¹ is (Ic), Z is alkylene and R² is heteroaryl.

In one embodiment, a is 2; M is —CH— or —CF—; Y is —C(O)—; R¹ is (Ia), Z is alkylene and R² is heteroaryl.

In another embodiment, a is 2; M is or —CF—; Y is —C(O)—; R¹ is (Ib), Z is alkylene and R² is heteroaryl.

In another embodiment, a is 2; M is —CH— or —CF—; Y is —O—; R¹ is (Ic); Z is alkylene and R² is heteroaryl.

In one embodiment, Y is —C(O)—; R¹ is (Ia); Z is alkylene and R² is pyridyl, thiazolyl, pyridazinyl or pyrimidinyl.

In another embodiment, Y is —C(O)—; is (Ib); Z is alkylene and R² is pyridyl, thiazolyl, pyridazinyl or pyrimidinyl.

In another embodiment, Y is —O—; R¹ is (Ic); Z is alkylene and R² is pyridyl, thiazolyl, pyridazinyl or pyrimidinyl.

In one embodiment, Y is —C(O)—; Z is alkylene; R² is heteroaryl and R¹ is:

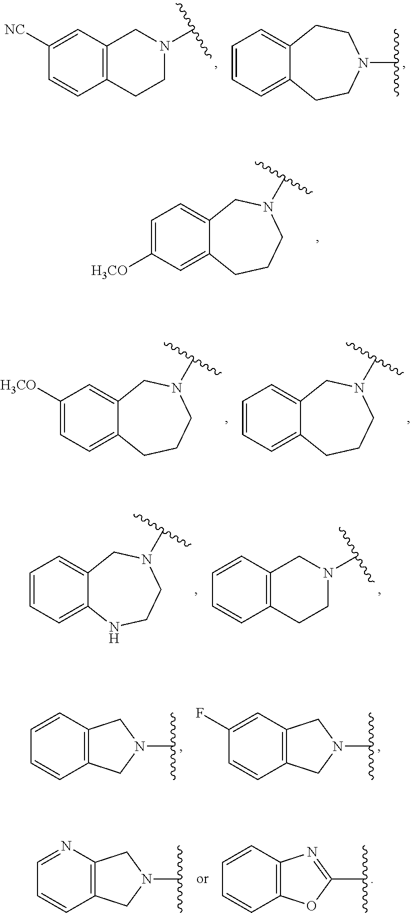

In another embodiment, Y is —O—; Z is alkylene; R² is heteroaryl and R¹ is:

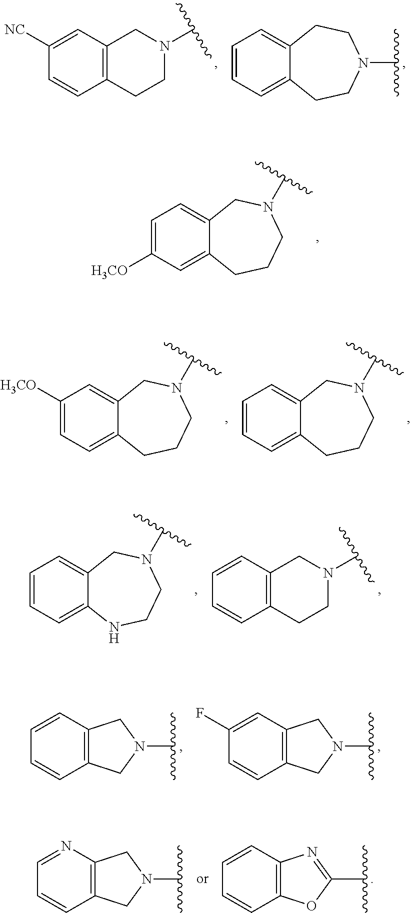

In one embodiment, Y is —C(O)—; Z is alkylene; R² is pyridyl, thiazolyl, pyridazinyl or pyrimidinyl and R¹ is:

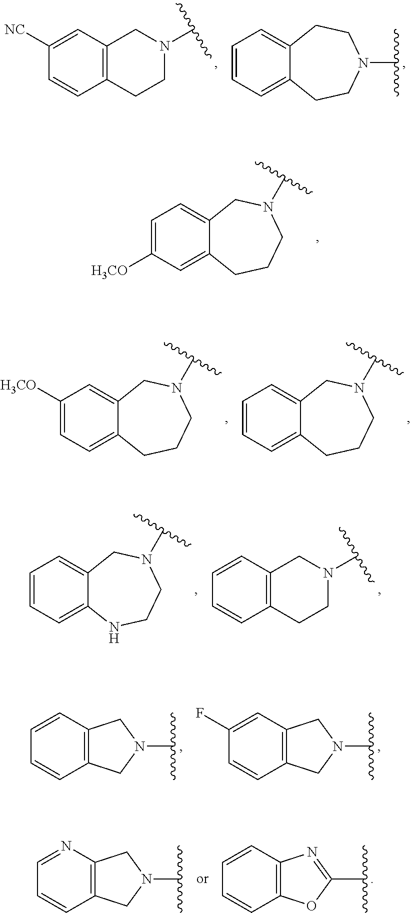

In another embodiment, Y is —O—; Z is alkylene; R² is pyridyl, thiazolyl, pyridazinyl or pyrimidinyl and R¹ is:

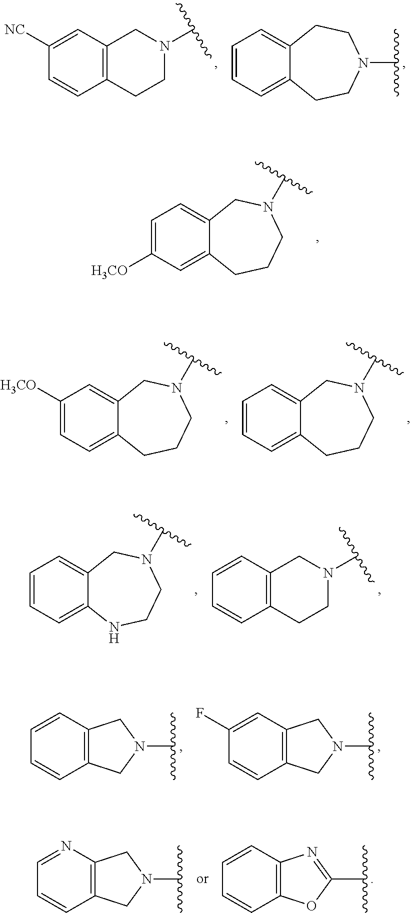

In one embodiment, Y is —C(O)—; Z is alkylene; R² is

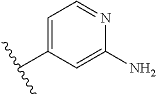

### and R¹ is:

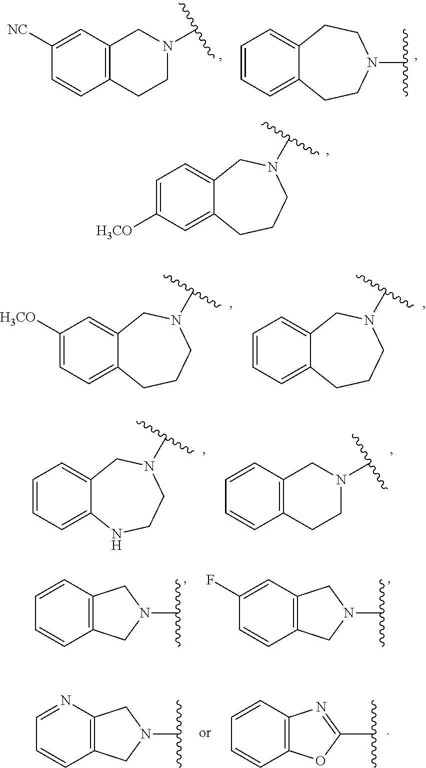

In another embodiment, Y is —O—; Z is alkylene; R² is

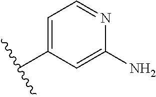

### and R¹ is:

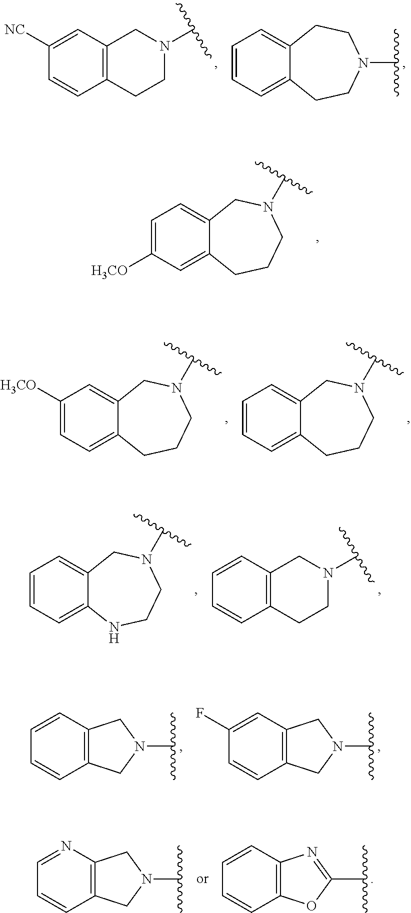

In one embodiment, M is —CH—.

In another embodiment, M is —C(halo)-.

In another embodiment, M is —CF—.

In one embodiment, a is 2.

In another embodiment, a is 2 and M is —CH— or —C(halo)-.

In one embodiment, b is 0.

In one embodiment, Z is alkylene.

In another embodiment, Z is —CH₂—.

In another embodiment, Z is —CH(CH₃)—.

In one embodiment, R² is heteroaryl.

In another embodiment, R² is amino-substituted heteroaryl.

In another embodiment, R² is aminopyridyl.

In another embodiment, R² is aminothiazolyl,

In one embodiment, the present invention includes compounds of formula being defined by any of the above embodiments or combinations thereof.

In another embodiment, for the Compounds of Formula (I), R¹, R², R⁴, M, Y, Z, a and b are selected independently from each other.

In another embodiment, a Compound of Formula (I) is in purified form.

Non-limiting illustrative examples of the Compounds of Formula (I) include the following compounds:

| Compound | | |
|---|---|---|
| No. | Structure | (M + H) |
| 1 | 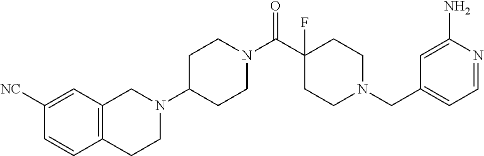 | 477 |
| 2 | 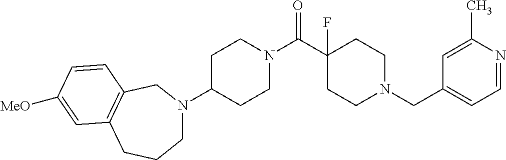 | 495 |
| 3 | 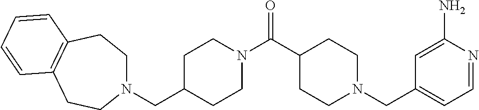 | 462 |
| 4 | 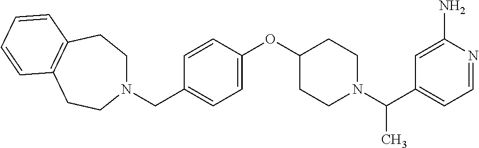 | 457 |
| 5 | 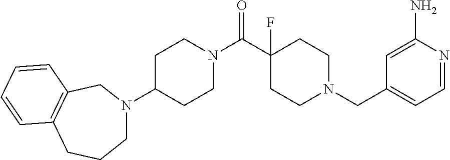 | 466 |
| 6 | 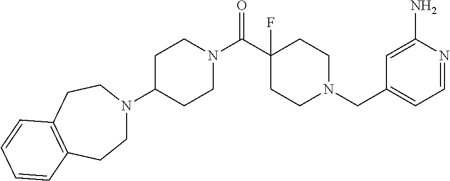 | 466 |
| 7 | 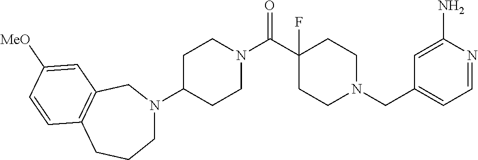 | 496 |
| 8 | 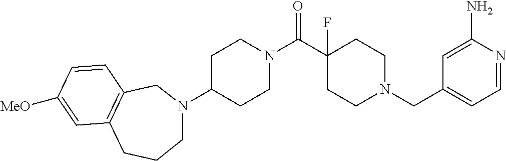 | 496 |
| 9 | 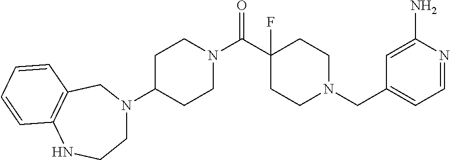 | 467 |
| 10 | 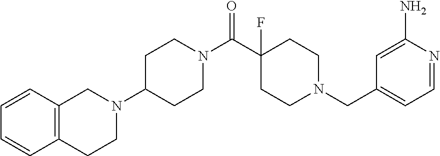 | 452 |
| 11 | 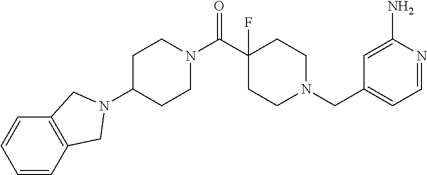 | 438 |
| 12 | 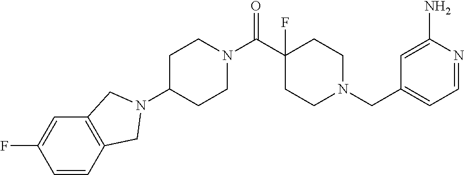 | 456 |
| 13 | 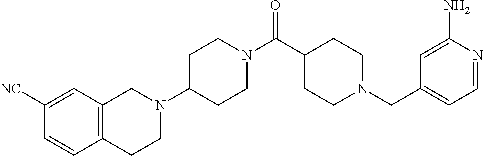 | 459 |
| 14 | 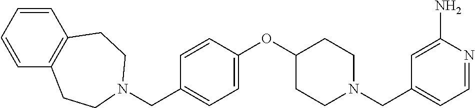 | 443 |
| 15 | 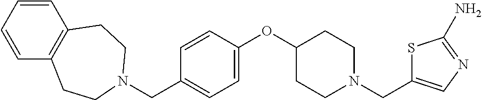 | 449 |
| 16 | 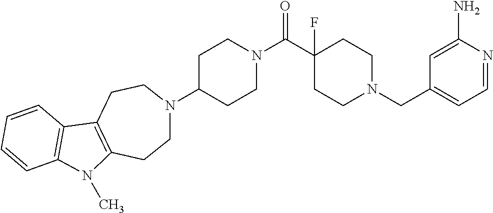 | 519 |
| 17 | 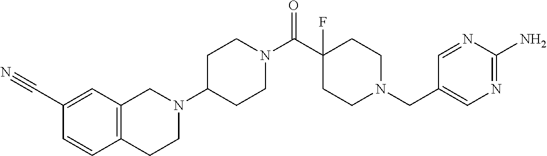 | 478 |
| 18 | 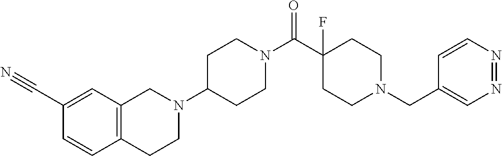 | 463 |
| 19 | 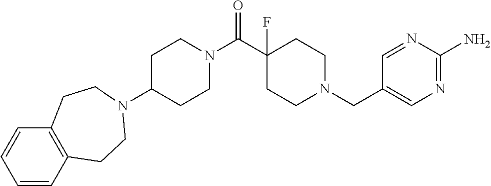 | 467 |
| 20 | 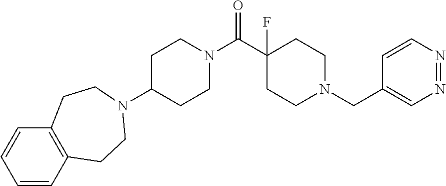 | 452 |
| 21 | 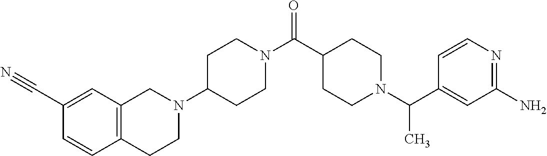 | 473 |
| 22 | 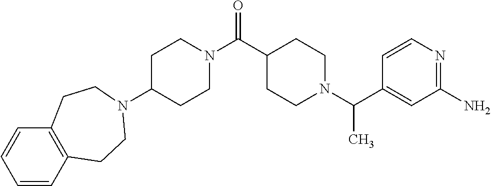 | 462 |
| 23 | 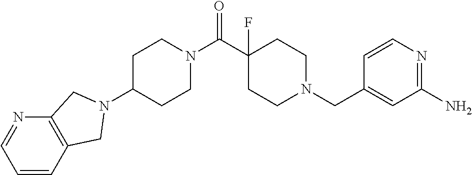 | 439 |
| 24 | 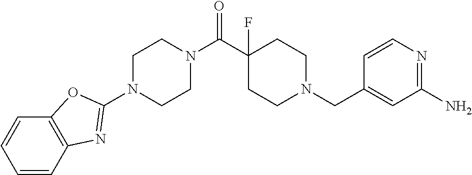 | 439 |
| 25 | 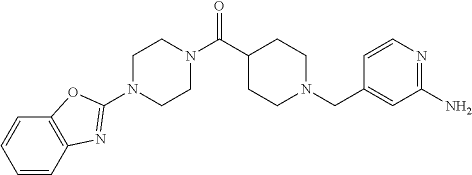 | 421 |

and pharmaceutically acceptable salts, solvates, esters and prodrugs thereof.

### Methods For Making The Compounds of Formula (I)

Methods useful for making the Compounds of Formula (I) are set forth in the Examples below and generalized in Schemes 1-6. Alternative synthetic pathways and analogous structures will be apparent to those skilled in the art of organic synthesis.

Scheme 1 shows methods useful for making the compounds of formula (I) via a convergent synthesis in which intermediate compounds AB and CD are coupled to provide compounds of formula ABCD, which correspond to the compounds of formula (I).

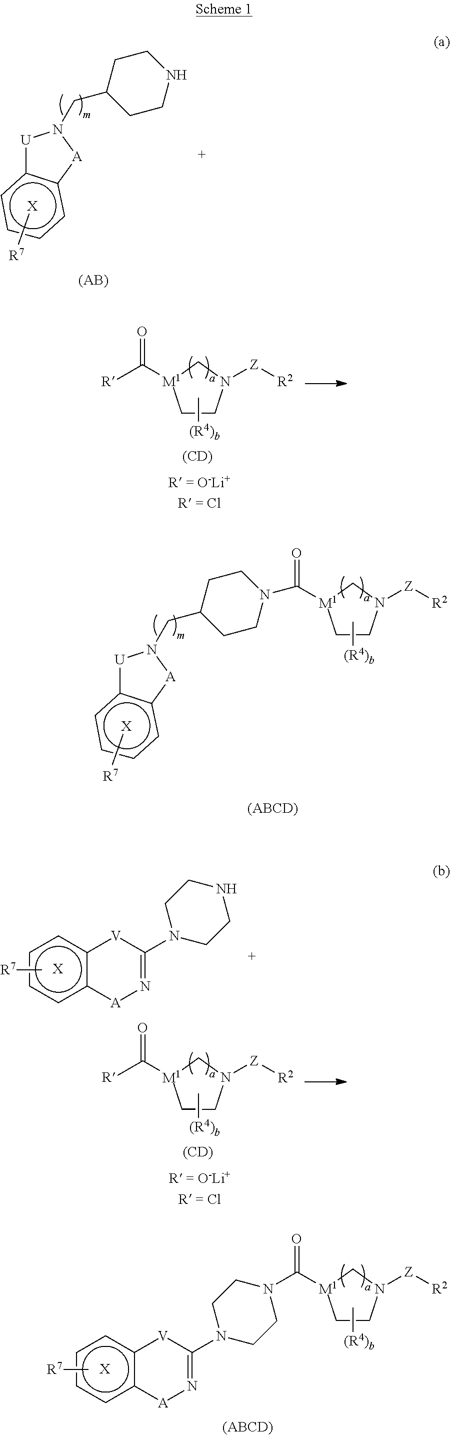

In Scheme 1(a), oxalyl chloride is used to convert lithium carboxylate CD to the corresponding acid chloride which is coupled with the AB moiety in the presence of diisopropylethyl amine. Alternatively, the corresponding lithium carboxylate CD can be directly coupled with AB using N-(3-dimethylaminopropyl)-N′-ethylcarbodiimide hydrochloride (EDC) and 1-hydroxy-benzotriazole (BtOH) as shown in Scheme 1(b). The synthesis of the AB group via a coupling of the A and B fragments is described in Scheme 6. Various examples of C and D fragments, as well as the methods employed in the synthesis of the CD portion, and the addition of C and then D fragments onto the initial AB fragment have been previously described in detail (e.g., International Publication No. WO 2002/32893).

Scheme 2 illustrates methods useful for the coupling of the C and D moities to make the intermediates of formula CD.

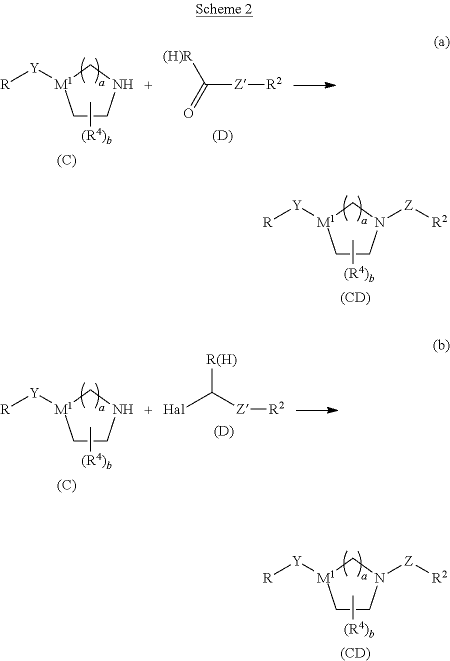

Scheme 2(a) shows a process by which the amine group of a C fragment can be coupled with an aldehyde or ketone D fragment via a reductive amination process using, for example, sodium triacetoxyborohydride. Scheme 2(b) shows a process by which the amine group of a C fragment can be coupled with an alkyl halide D fragment, particularly an alkylbromide or an alkylchloride, using a carbonate base such as potassium carbonate.

Scheme 3 shows a linear synthesis of the compounds of formula (I) involving first assembling the ABC portion through the coupling of A and BC fragments, then adding on the D fragment to make the compounds of formula ABCD, which correspond to the compounds of formula (I).

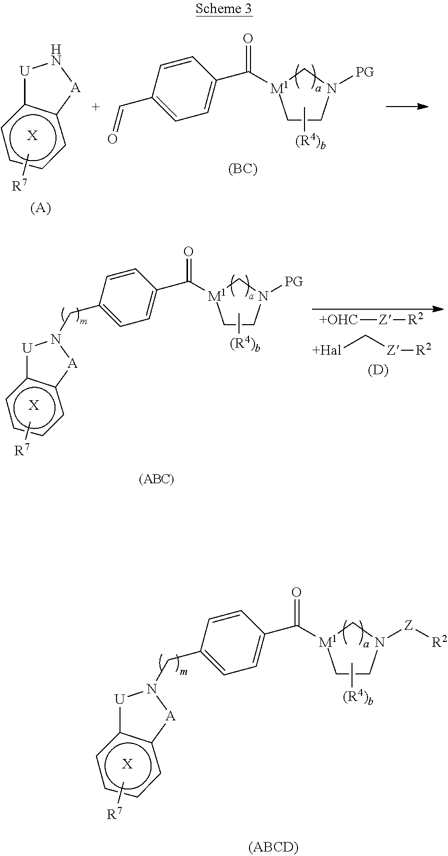

Fragment A can be coupled with BC via a reductive amination process using sodium triacetoxyborohydride and the resulting ABC unit can be subsequently coupled with a D fragment using the methods described above in Scheme 2. Alternative methods for the coupling of A with BC are set forth below in Scheme 6.

Fragment A can be obtained commercially available or can be prepared using known methods, such as those described, for example, in Liebigs Annalen der Chemie (1979), 3, 328-333; Chemical & Pharmaceutical Bulletin (1975), 23(9), 1917-27: and Journal of Medicinal Chemistry (2005), 48(10), 3586-3604. The BC fragments used in preparing the compounds described are either available from commercial suppliers or can be prepared using known methods, such as those described, for example, in Bioorganic & Medicinal Chemistry (2005) 13(3), 725-734, and Journal of Medicinal Chemistry (1995), 38(23), 4634-4636.

Scheme 4 shows alternate methods of linear synthesis for making the compounds of formula (I) by first assembling the ABC moiety via a coupling of an AB fragment and a C fragment, then adding a D fragment onto the ABC moiety. This provides the compounds of formula ABCD, which correspond to the compounds of formula (I).

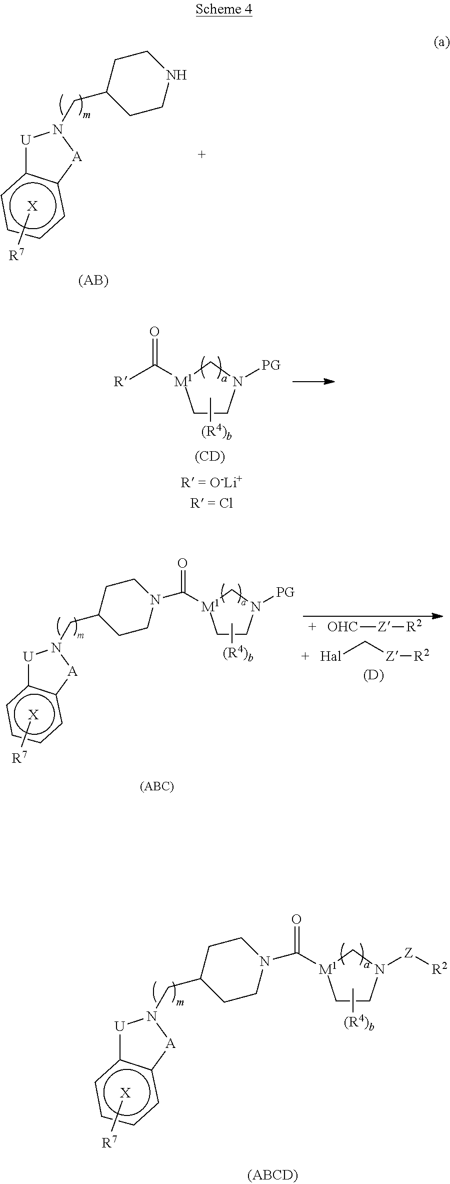

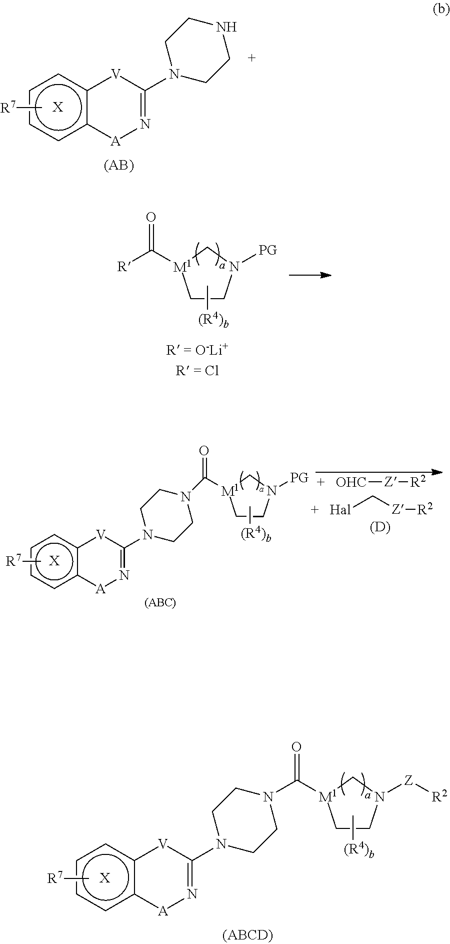

In Scheme 4(a), the coupling of the AB and C fragments can be carried out using the methods describe in Scheme 1 for coupling AB with CD to provide the compounds of formula ABCD. Scheme 4(b) illustrates how the methods described in Scheme 2 for the coupling of C with D can be used to couple ABC with D in order to make the compounds of formula ABCD.

Scheme 5 shows yet another linear synthesis method useful for making the compounds of formula (I). This method entails first assembling the BCD moiety by the coupling a B fragment with a CD fragment, then adding an A fragment onto group BCD to provide the compounds of formula ABCD, which correspond to the compounds of formula (I).

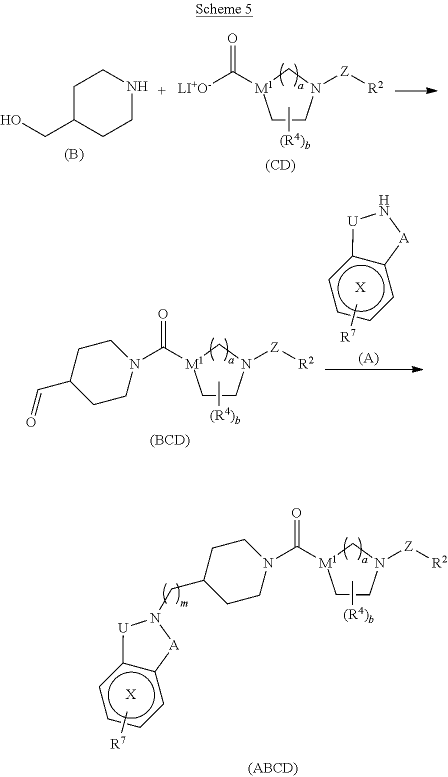

The coupling of fragments B and CD can be carried out using the methods depicted in Scheme 1 for coupling AB with CD. The resulting coupled product contains a primary hydroxy group which is oxizided to provide the aldehyde containing BCD moiety. BCD is then coupled with fragment A using a reductive amination process employing sodium triacetoxyborohydride. Alternative methods useful for coupling A with BCD are shown below in Scheme 6.

Scheme 6 shows alternative methods useful for linking fragments A and B. These methods can also be used to couple A with BC or A with BCD.

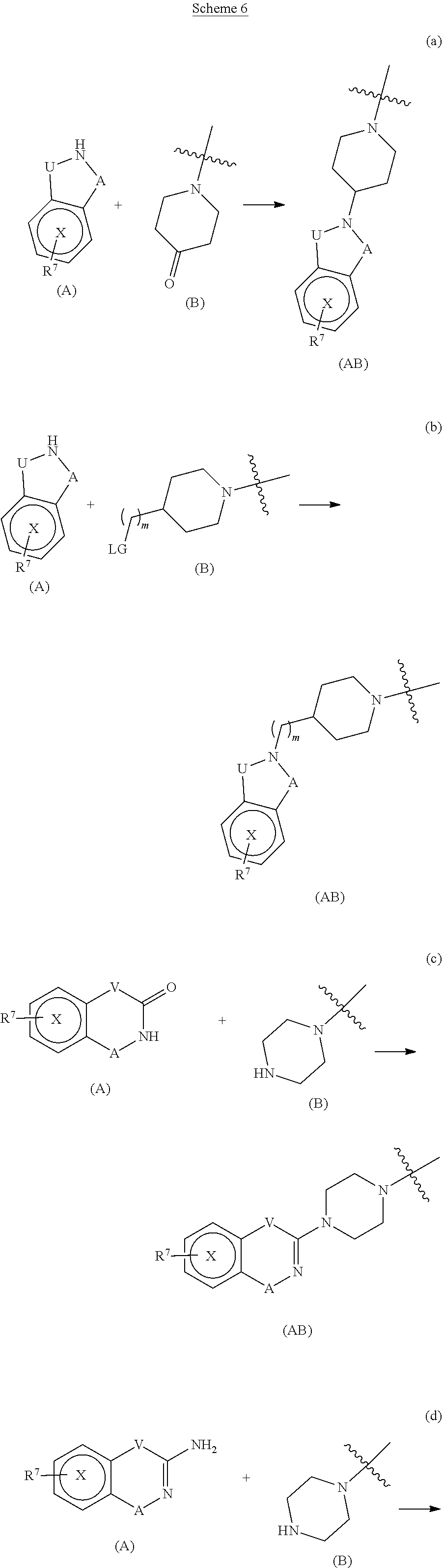

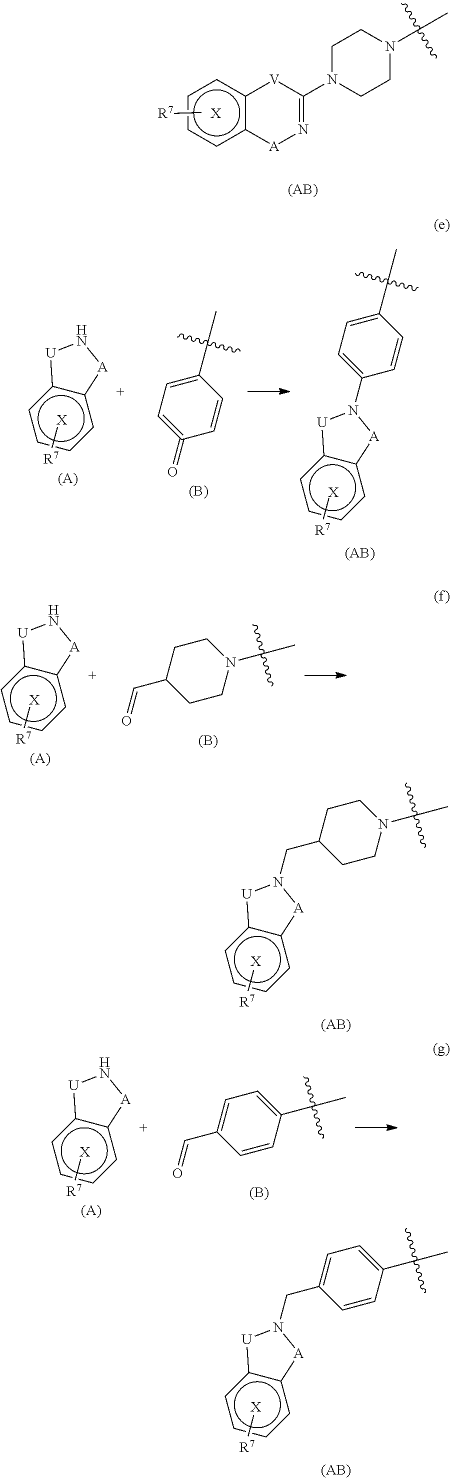

Fragment A can be coupled with a piperidine B fragment using various methods, including, but not limited to a reductive amination process (Schemes 6(a) and 6(c)) or a nucleophilic displacement of a halogen or other known leaving group (LG) such as a mesylate, tosylate or triflate (Scheme 6(b)). An amidine linkage between fragments A and B (Scheme 6(d)) can be formed using known methods, such as those described in Australian Journal of Chemistry (2002), 55(9), 565-576, and International Publication No. WO 04/000847. When fragment B is an aromatic ring, N-arylation can be performed as described in Scheme 6(e) via nucleophilic aromatic substitution using aryl halides or aryl mesylates, tosylates or triflates as the leaving group (LG), or using a Cu, Zn—Cu or Pd catalyzed cross-coupling reaction between the N atom in fragment A and the corresponding aryl-halides, -mesylates, -tosylates or -triflates present as group LG. When there is a methylene linker between the A and B fragments, moiety AB can be formed via nucleophilic displacement of a halogen, or other known leaving groups such as mesylates, tosylates or triflates, at the benzylic position in fragment B, or by a reductive amination process from the corresponding aldehydes at fragment B (Schemes 6(f) and 6(g)).

The starting materials and reagents depicted in Schemes 1-6 are either available from commercial suppliers such as Sigma-Aldrich (St. Louis, Mo.) and Acros Organics Co. Fair Lawn, N.J.), or can be prepared using methods well-known to those of skill in the art of organic synthesis.

One skilled in the art will recognize that the synthesis of compounds of Formula (I) may require the need for the protection of certain functional groups (i.e., derivatization for the purpose of chemical compatibility with a particular reaction condition). Suitable protecting groups for the various functional groups of the compounds of formula (I) and methods for their installation and removal may be found in Greene et al., Protective Groups in Organic Synthesis, Wiley-Interscience, New York, (1999).

### EXAMPLES

The following examples exemplify illustrative examples of compounds of the present invention and are not to be construed as limiting the scope of the disclosure. Alternative mechanistic pathways and analogous structures within the scope of the invention may be apparent to those skilled in the art.

### General Methods

The starting materials and reagents used in preparing compounds described are either available from commercial suppliers such as Aldrich Chemical Co. (Wisconsin, USA) and Acros Organics Co. (New Jersey, USA) or were prepared using methods well-known to those skilled in the art of organic synthesis. All commercially purchased solvents and reagents were used as received. LCMS analysis was performed using an Applied Biosystems API-100 mass spectrometer equipped with a Shimadzu SCL-10A LC column: Altech platinum C18, 3 um, 33 mm×7 mm ID; gradient flow: 0 minutes, 10% CH₃CN; 5 minutes, 95% CH₃CN; 7 minutes, 95% CH₃CN; 7.5 minutes, 10% CH₃CN; 9 minutes, stop. Flash column chromatography was performed using Selecto Scientific flash silica gel, 32-63 mesh. Analytical and preparative TLC was performed using Analtech Silica gel GF plates. Chiral HPLC was performed using a Varian PrepStar system equipped with a Chiralpak OD column (Chiral Technologies).

### Example 1

### Preparation of Compound 1

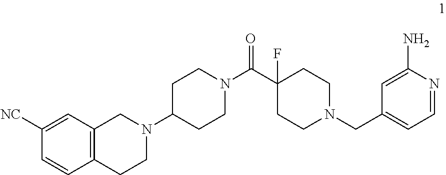

### Step 1—Synthesis of Compound 1c

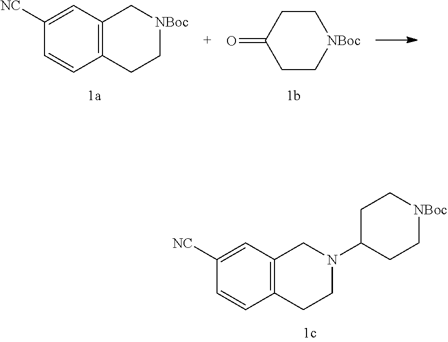

Sodium triacetoxyborohydride (2.57 g, 12.12 mmol, 1.8 eq) was added to a stirred solution of compound 1b (1.75 g, 8.78 mmol, 1.3 eq) and compound 1a (1.07 g, 636 mmol) in dry DCM (200 mL) at room temperature, and the resulting mixture was allowed to stir for 15 hours. Then the mixture was diluted with DCM and treated with saturated aqueous K₂CO₃ solution. The layers were separated and the aqueous was extracted with DCM. The combined organic extracts were dried over MgSO4, filtered and concentrated in vacuo to provide a residue that was purified using flash column chromatography on silica gel (DCM:0.4N NH₃ in MeOH 95:5) to provide compound 1c (1.71 g, 74%) as a yellow oil.

### Step 2—Synthesis of Compound 1d

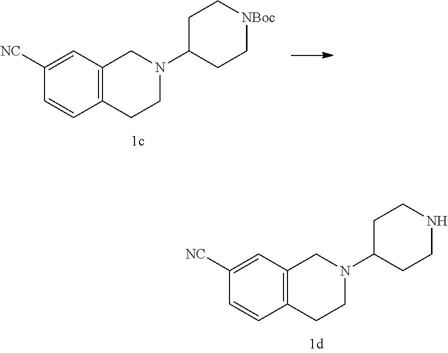

A solution of compound 1c (1.71 g, 6.76 mmol) in a mixture of dichloromethane (90 mL) and trifluoroacetic acid (15 mL) was allowed to stir at room temperature for 2 hours. The reaction mixture was then cooled to 0° C. and basified slowly with 10% aqueous ammonia. The resulting mixture was extracted with dichloromethane (2×150 mL) and the combined organic extracts were dried over MgSO₄, filtered and concentrated in vacuo to provide piperidine 1d (0.850 g 70%) as a pale yellow foam.

### Step 3—Synthesis of Compound 1f

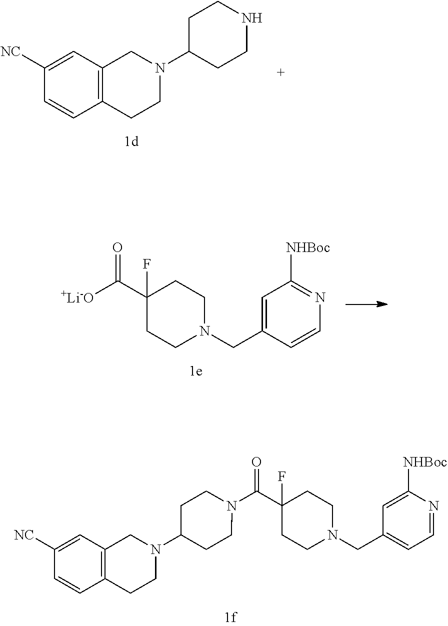

A sealed tube (15 mL) was charged with compound 1d (190 mg, 0.79 mmol), compound 1e (made as described in US Patent Publication No. 2002/32893) (283 mg, 0.79 mmol, 1.0 eq), EDC (227 mg, 1.19 mmol, 1.5 eq), BtOH (160 mg. 1.19 mmol, 1.5 eq) and dichloromethane (6 mL). The resulting mixture was heated at 60° C. for 15 hours, then cooled to room temperature, diluted with dichloromethane (50 mL) and washed with 1 N aqueous NaOH (30 mL). The layers were separated and the aqueous was extracted with dichloromethane (2×25 mL). The combined organic extracts were dried and concentrated in vacuo to provide an oil that was purified using preparative TLC (SiO₂, dichloromethane: 0.4 N NH₃ in MeOH 92:8) to provide 1f (116 mg, 31%) as an orange solid.

### Step 4—Synthesis of Compound 1

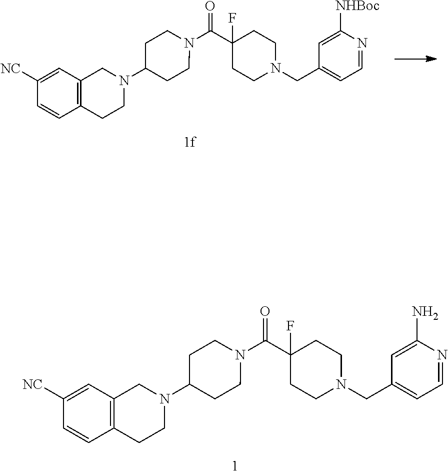

Trifluoroacetic acid (3 mL) was added to a solution of Roc-protected aminopyridine if 16 mg, 0.20 mmol) in DCM (10 mL). The resulting solution was stirred under a nitrogen atmosphere for 3 hours, then cooled to 0° C. and carefully basified with 15% aqueous ammonia solution. The layers were separated and the aqueous extracted with DCM (1×50 mL). The combined organic extracts were dried over MgSO₄ and concentrated in vacuo to provide compound 1 (80 mg, 85%) as a colorless foam. MS: (M+1-1) 477.

### Example 2

### Preparation of Compound 2

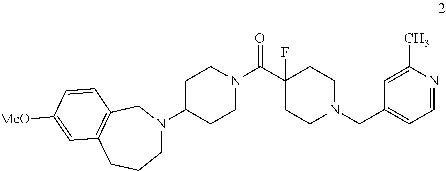

Step 1—Synthesis of Compound 2b

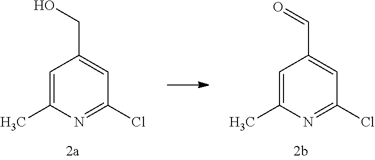

Manganese (IV) oxide (4.6 g, 52.9 mmol, 8.0 eq) was added to a stirred solution of compound 2a in dry chloroform (20 mL) and the resulting mixture was allowed to stir for 144 hours at room temperature. The reaction mixture was filtered and concentrated in vacuo to provide compound 2b (781 mg, 62%) as yellow crystals.

### Step 2—Synthesis of Compound 2c

Using the method described in Step 2 of Example 1, compound 2b was converted to compound 2e.

### Step 3—Synthesis of Compound 2d

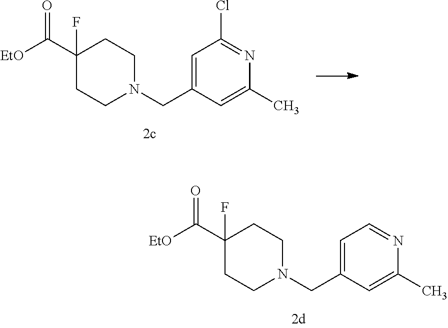

Triethylamine (6.9 mL, 49.5 mmol, 5.0 eq), formic acid (1.8 mL, 49.5 mmol, 5.0 eq) and bis-1,1′(diphenylphosphino) ferrocenedichloro palladium (II) (520 mg, 0.64 mmol, 6.7 mol %) were added to a stirred solution of compound 2e (2.996 g. 9.52 mmol) in dry DMF (50 mL) at room temperature. The resulting mixture was heated at 70° C. and allowed to stir at this temperature for 2.5 hours, then the reaction mixture was cooled to room temperature and concentrated in vacuo. The brown residue obtained was purified using flash column chromatography on silica gel to provide compound 2d (2.65 g, 99%) as a dark brown solid.

### Step 4—Synthesis of Compound 2f

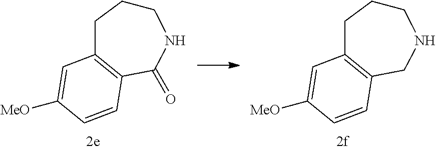

A solution of compound 2e (1.00 g, 5.23 mmol) in dry THF (25 mL) was added to a stirred suspension of lithium aluminum hydride (900 mg, 23.7 mmol, 4.53 eq) in dry THF (30 mL) at 0° C. The resulting mixture was heated to reflux and allowed to stir at this temperature for 15 hours, then cooled to room temperature and quenched with aqueous 25% NaOH. A saturated solution of Na—K tartrate (200 mL) was added and the resulting solution was allowed to stir at room temperature for 2 hours. The mixture was then extracted with DCM (2×200 mL), the combined organic extracts were dried over MgSO₄, filtered and concentrated in vacuo to provide compound 2f (903 mg, 97%) as a colorless oil.

### Step 5—Synthesis of Compound 2

Using the methods described in Steps 1, 2 and 3 of Example 1, compound 2 was prepared from compound 2f. MS: (M+1) 495.

### Example 3

### Preparation of Compound 3

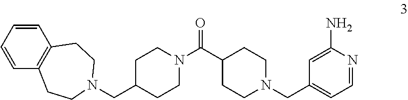

Step 1—Synthesis of Compound 3c

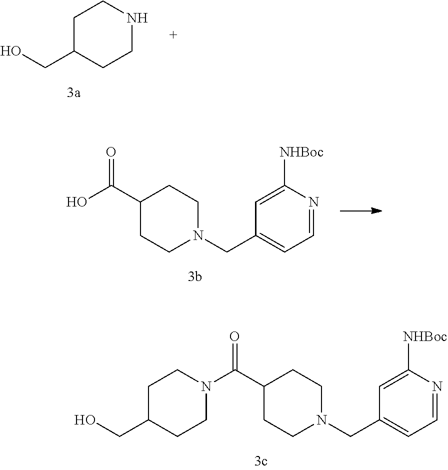

To a solution of compound 3b (4.0 g, 0.012 mol) and compound 3a (4.1 g, 0.035 mol) in DCM (200 mL) was added EDC (6.8 g, 0.035 mol), HOBt (4.8 g, 0.035 mol) and Et₃N (3.6 g, 0.035 mol). The resulting reaction was allowed to stir at room temperature for 18 hours, then diluted with DCM, washed with 1N NaOH and brine, dried over MgSO₄, filtered and concentrated in vacuo. The residue obtained was purified using flash column chromatography on silica gel (MeOH/DCM, 1:10) to provide compound 3c (3.9 g, 79%).

Step 2—Synthesis of Compound 3d

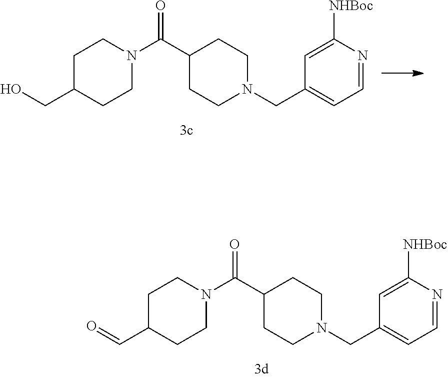

To a solution of compound 3c (2.0 g, 4.63 mmol) in DCM (20 mL) was added pyridine (1.3 mL, 16.2 mmol) and Dess-Martin peridinane (7.9 g, 11.6 mmol). The resulting reaction was allowed to stir at room temperature for 12 hours, then saturated aqueous NaHCO₃ and saturated aqueous NaS₂O₃ was added and the resulting solution was allowed to stir for 20 minutes. The mixture was then extraced with DCM and the DCM was dried over MgSO₄, filtered and concentrated in vacuo. The resulting residue was purified using flash column chromatography on silica gel (MeOH/DCM, 1:30) to provide compound 3d (1.1 g, 55%).

### Step 3—Synthesis of Compound 3

Using the methods described in Step 1 and Step 4 of Example compound 3 was prepared from compound 3d. MS: (M+1) 462.

### Example 4

### Preparation of Compound 4

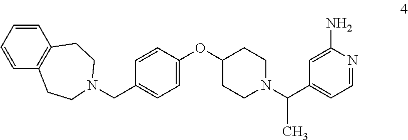

### Step 1—Synthesis of Compound 4c

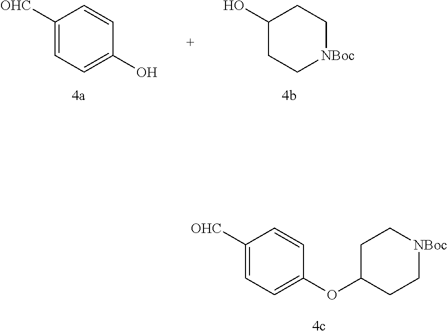

To a stirred solution of compound 4a (7.32 g, 0.06 mol), compound 4b (6.0 g, 0.03 mol) and triphenylphosphine (11.8 g, 0.045 mol) in dry THF (100 mL) was slowly added diethylazodicarboxylate (7.83 g, 0.045 mol). The resulting reaction heated to 65° C. and allowed to stir at this temperature for 12 hours, then cooled to room temperature and diluted with EtOAc. The resulting solution was washed with 1 N NaOH (2×) then brine, dried over MgSO₄, filtered and concentrated in vacuo. The resulting residue was purified using flash column chromatography on silica gel (EtOAc:Hexanes/1:2, then 1:1) to provide compound 4c (6.2 g, 68% yield).

### Step 2—Synthesis of Compound 4e

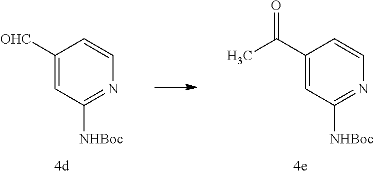

To a solution of compound 4d (4.32 g, 0.02 mol) in THF (50 mL) at 0° C. was slowly added MeMgBr (3.0 M in Et₂O, 0.05 mol, 16.3 mL) slowly. The reaction was warmed up to room temperature, stirred at this temperature for 2 hours, then quenched with saturated aqueous NH₄Cl solution. The resulting solution was extracted with EtOAc and the organic layer was washed with saturated NH₄Cl, dried over MgSO₄, filtered and concentrated in vacuo. The resulting oil was dissolved in DCM (50 mL) and to the resulting solution was added Dess-Martin periodinane (10.3 g, 0.025 mol) and the resulting reaction was allowed to stir at room temperature for 2 hours. The reaction mixture was quenched with 10% aqueous Na₂S₂O₃, followed by 1 N aqueous NaOH, and the resulting solution was diluted with DCM. The organic layer was dried over MgSO₄, filtered and concentrated in vacuo to provide compound 4e as a yellow solid.

### Step 3—Synthesis of Compound 4

Using the methods described in Steps 1 and 4 of Example 1, compound 4 was prepared from compound 4e. MS: (M+1) 457.

### Example 5

### H₃ Receptor Binding Assay

The source of H₃ receptors used was recombinant human receptor, expressed in HEK-293 (human embryonic kidney) cells. The membranes were frozen and stored at −70° C. until needed.

Compounds of the invention to be tested were dissolved in DMSO and then diluted into the binding buffer (50 mM Tris, pH 7.5) such that the final concentration was about 2 μg/ml with 0.1% DMSO. Recombinant human receptor membranes were then added (5 μg of protein) to the reaction tubes. The reaction was initiated via the addition of 3 nM [³H]R-α-methyl histamine (8.8 Ci/mmol) or 3 nM [³H]N^{α}-methyl histamine (80 Ci/mmol) and continued under incubation at 30° C. for 30 minutes. Bound ligand was separated from unbound ligand by filtration, and the amount of radioactive ligand bound to the membranes was quantitated using liquid scintillation spectrometry. All incubations were performed in duplicate and the standard error was always less than 10%. Compounds that inhibited more than 70% of the specific binding of radioactive ligand to the receptor were serially diluted to determine a Kᵢ (nM).

Using this method it was shown that the compounds of the present invention demonstrate Kᵢ values of from about 1 nM to about 100 nM at the recombinant human H₃ receptor.

### Example 6

### In Vivo Effect of Compounds of the Invention on Glucose Levels in Diabetic Mice

Five-week-old male ICR mice (which can be purchased for example, from Taconic Farm, Germantown, N.Y.) are placed on a “western diet” containing 45% (kcal) fat from lard and 0.12% (w/w) cholesterol. After 3 weeks of feeding, the mice are injected once with low dose streptozocin (STZ, ip 75-100 mg/kg) to induce partial insulin deficiency. Two weeks after receiving the STZ injection, the animals that have developed type 2 diabetes and display hyperglycemia, insulin resistance, and glucose intolerance are placed in one of three groups: (1) a non-treated control group, (2) a group treated with rosiglitazone (5 mg/kg/day in diet); or (3) a group treated with a compound of the present invention (10/mg/kg in diet). The animals in groups (2) and (3) are treated daily at the designated dosages for total period of four weeks. The glucose levels in the three groups can then be compared to determine the effectiveness of the compounds of the invention in lowering glucose levels in the diabetic animals.

### Example 7

### In Vivo Effect of Compounds of the Invention on Glucose Levels in Diabetic Rats

Adult, diabetic, Goto-Kakizaki rats (14 weeks old) are tested for non-fasting glucose levels using a glucometer. Rats with glucose levels between 130 and 370 mg/dl are then randomized into treatment (N=10) and control (N=10) groups. Animals in the treatment group are administered a compound of the present invention in their food chow at a dose of 10 mg/kg/day. After one week of treatment, blood is collected via tail snip and the non-fasting glucose level is measured using a glucometer. The glucose levels of the animals in the treated group are compared to the glucose levels of the animals in the control group to determine the effectiveness of the test compound in lowering glucose levels in the diabetic animals.

### Example 8

### In Vivo Effect of Compounds of the Invention on Obese Mice

Five-week old mice (20-25 Jackson lab, Maine) were maintained in individual cages at 22° Con a 12:12 hour light/dark cycle with lights on at 1100. Mice (n=12 per group) were balanced by body weight and food intake while on a standard laboratory chow (Teklad, formulation 2001) after an oral dosing with vehicle (20% hpbcd; 1 mL/kg). The following week, mice were switched from a chow diet into a high fat diet HF (Research Diets, New Brunswick, N.J., formulation #D 12451, 4.7 kcal/g, comprised of 45% fat, 35% CHO, 20% protein). Daily oral gavage of vehicle or compound in vehicle occurred about the same time each day, approximately 1 hour before dark onset. Each day, HF pellets were pre-weighed and placed in the home cage immediately following daily oral gavage. Body weight and HF food intake were monitored daily for 4 days.

Using this method, it was demonstrated that selected illustrative compounds of the present invention, when administered at doses 1-30 mg/kg/day by oral gavage, significantly reduced body weights relative to control mice. Accordingly, the compounds of the present invention are useful for treating obesity.

### Uses of the Compounds of Formula (I)

The Compounds of Formula (I) are useful in human and veterinary medicine for treating or preventing a Condition in a patient. In accordance with the invention, the Compounds of Formula (I) can be administered to a patient in need of treatment or prevention of a Condition.

Accordingly, in one embodiment, the invention provides methods for treating a Condition in a patient comprising administering to the patient an effective amount of one or more compounds of Formula (I) or a pharmaceutically acceptable salt, solvate, ester or prodrug thereof. In addition, the present invention provides methods for treating or preventing Condition in a patient, comprising administering to the patient one or more Compounds of Formula (I) and an additional therapeutic agent that is not a Compound of Formula (I), wherein the amounts administered are together effective to treat or prevent the Condition.

In one embodiment, the compounds of the present invention can be ligands for the histamine H₃ receptor. In another embodiment, the compounds of the present invention can also be described as antagonists of the H₃ receptor, or as H₃ antagonists.

### Treating or Preventing Allergy

The Compounds of Formula (I) are useful for treating or preventing allergy in a patient.

Accordingly, in one embodiment, the present invention provides a method for treating allergy in a patient, comprising administering to the patient an effective amount of one or more Compounds of Formula (I).

Non-limiting examples of allergy treatable or preventable using the present methods include Type I hypersensitivity reactions, Type II hypersensitivity reactions, Type III hypersensitivity reactions, Type IV hypersensitivity reactions, food allergies, allergic lung disorders, allergic reaction to a venomous sting or bite; mold allergies, environmental-related allergies (such allergic rhinitis, grass allergies and pollen allergies), anaphlaxis and latex allergy.

In one embodiment, the allergy is an environmental-related allergy.

### Treating or Preventing Allergy-Induced Airway Response

The Compounds of Formula (I) are useful for treating or preventing allergy-induced airway response in a patient.

Accordingly, in one embodiment, the present invention provides a method for treating allergy-induced airway response in a patient, comprising administering to the patient an effective amount of one or more Compounds of Formula (I).

Non-limiting examples of allergy-induced airway response treatable or preventable using the present methods include upper airway responses.

In one embodiment, the allergy-induced airway response is an upper airway response.

### Treating or Preventing Congestion

The Compounds of Formula (I) are useful for treating or preventing congestion in a patient.

Accordingly, in one embodiment, the present invention provides a method for treating congestion in a patient, comprising administering to the patient an effective amount of one or more Compounds of Formula (I).

Non-limiting examples of congestion treatable or preventable using the present methods include nasal congestion and all types of rhinitis, including atrophic rhinitis, vasomotor rhinitis, gustatory rhinitis and drug induced rhinitis.

In one embodiment, the congestion is nasal congestion.

### Treating or Preventing a Neurological Disorder

The Compounds of Formula (I) are useful for treating or preventing a neurological disorder in a patient. The term “neurological disorder,” as used herein, refers to a disorder of any part of the central nervous system, including, but not limited to, the brain, nerves and spinal cord.

Accordingly, in one embodiment, the present invention provides a method for treating a neurological disorder in a patient, comprising administering to the patient an effective amount of one or more Compounds of Formula (I).

Non-limiting examples of neurological disorders treatable or preventable using the present methods include pain, hypotension, meningitis, a movement disorder (such as Parkinson's disease or Huntington's disease), delirium, dementia, Alzheimer's disease, a demyelinating disorder (such as multiple sclerosis or amyotrophic lateral sclerosis), aphasia, a peripheral nervous system disorder, a seizure disorder, a sleep disorder, a spinal cord disorder, stroke, a congnition deficit disorder (such as attention deficit hyperactivity disorder (ADHD)), hypo and hyperactivity of the central nervous system (such as agitation or depression) and schizophrenia.

In one embodiment, the neurological disorder is a sleep disorder.

In another embodiment, the neurological disorder is a movement disorder.

In another embodiment, the neurological disorder is Alzheimer's disease.

In yet another embodiment, the neurological disorder is schizophrenia.

In another embodiment, the neurological disorder is hypotension.

In one another embodiment, the neurological disorder is depression.

In another embodiment, the neurological disorder is a cognition deficit disorder.

In a further embodiment, the neurological disorder is ADHD, which can be present in an adult or a child.

In one embodiment, the sleep disorder is hypersomnia, somnolence or narcolepsy.

In another embodiment, the movement disorder is Parkinson's disease or Huntington's disease.

In one embodiment, the neurological disorder is pain.

Non-limiting examples of pain treatable or preventable using the present methods include acute pain, chronic pain, neuropathic pain, nociceptive pain, cutaneous pain, somatic pain, visceral pain, phantom limb pain, cancer pain (including breakthrough pain), pain caused by drug therapy (such as cancer chemotherapy), headache (including migraine, tension headache, cluster headache, pain caused by arthritis, pain caused by injury, toothache, or pain caused by a medical procedure (such as surgery, physical therapy or radiation therapy).

In one embodiment, the pain is neuropathic pain.

In another embodiment, the pain is cancer pain.

In another embodiment, the pain is headache.

### Treating or Preventing a Cardiovascular Disease

The Compounds of Formula (I) are useful for treating or preventing a cardiovascular disease in a patient.

Accordingly, in one embodiment, the present invention provides a method for treating a cardiovascular disease in a patient, comprising administering to the patient an effective amount of one or more Compounds of Formula (I).

Examples of cardiovascular diseases treatable or preventable using the present methods include, but are not hinted to, an arrhythmia, an atrial fibrillation, a supraventricular tachycardia, arterial hypertension, arteriosclerosis, coronary artery disease, pulmonary artery disease, a cardiomyopathy, pericarditis, a peripheral artery disorder, a peripheral venous disorder, a peripheral lymphatic disorder, congestive heart failure, myocardial infarction, angina, a valvular disorder or stenosis.

In one embodiment, the cardiovascular disease is atherosclerosis.

In another embodiment, the cardiovascular disease is coronary artery disease.

### Treating or Preventing a Gastrointestinal Disorder

The Compounds of Formula (I) are useful for treating or preventing a gastrointestinal disorder in a patient.

Accordingly, in one embodiment, the present invention provides a method for treating a gastrointestinal disorder in a patient, comprising administering to the patient an effective amount of one or more Compounds of Formula (I).

Examples of gastrointestinal disorders treatable or preventable using the present methods include, but are not limted to, hyper or hypo motility of the GI tract, acidic secretion of the GI tract, an anorectal disorder, diarrhea, irritable bowel syndrome, dyspepsis, gastroesophageal reflux disease (GERD), diverticulitis, gastritis, peptic ulcer disease, gastroenteritis, inflammatory bowel disease, a malabsorption syndrome or pancreatitis.

In one embodiment, the gastrointestinal disorder is GERD.

In another embodiment, the gastrointestinal disorder is hyper or hypo motility of the GI tract.

### Treating or Preventing an Inflammatory Disease

The Compounds of Formula (I) are useful for treating or preventing an inflammatory disease in a patient.

Accordingly, in one embodiment, the present invention provides a method for treating an inflammatory disease in a patient, comprising administering to the patient an effective amount of one or more Compounds of Formula (I).

### Treating or Preventing Non-Alcoholic Fatty Liver Disease

The Compounds of Formula (I) are useful for treating or preventing non-alcoholic fatty liver disease in a patient.

Accordingly, in one embodiment, the present invention provides a method for treating non-alcoholic fatty liver disease in a patient, comprising administering to the patient an effective amount of one or more Compounds of Formula (I).

### Treating or Preventing a Metabolic Disorder

The Compounds of Formula (I) can be useful for treating a metabolic disorder. Accordingly, in one embodiment, the invention provides methods for treating a metabolic disorder in a patient, wherein the method comprises administering to the patient an effective amount of one or more Compounds of Formula (I), or a pharmaceutically acceptable salt, solvate, ester or prodrug thereof.

Examples of metabolic disorders treatable include, but are not limited to, metabolic syndrome (also known as “Syndrome X”), impaired glucose tolerance, impaired fasting glucose, dyslipidemia, hypercholesterolemia, hyperlipidemia, hypertriglyceridemia, low HDL levels, hypertension, phenylketonuria, post-prandial lipidemia, a glycogen-storage disease, Gaucher's Disease, Tay-Sachs Disease, Niemann-Pick Disease, ketosis and acidosis.

In one embodiment, the metabolic disorder is hypercholesterolemia.

In another embodiment, the metabolic disorder is hyperlipidemia.

In another embodiment, the metabolic disorder is hypertriglyceridemia.

In still another embodiment, the metabolic disorder is metabolic syndrome.

In a further embodiment, the metabolic disorder is low HDL levels.

In another embodiment, the metabolic disorder is dyslipidemia.

### Treating or Preventing Obesity and Obesity-Related Disorders

The Compounds of Formula (I) can be useful for treating obesity or an obesity-related disorder. Accordingly, in one embodiment, the invention provides methods for treating obesity or an obesity-related disorder in a patient, wherein the method comprises administering to the patient an effective amount of one or more Compounds of Formula (I), or a pharmaceutically acceptable salt, solvate, ester or prodrug thereof.

### Methods For Treating or Preventing Diabetes

The Compounds of Formula (I) are useful for treating or preventing diabetes in a patient. Accordingly, in one embodiment, the present invention provides a method for treating diabetes in a patient, comprising administering to the patient an effective amount of one or more Compounds of Formula (I).

Examples of diabetes treatable or preventable using the Compounds of Formula (I) include, but are not limted to, type I diabetes (insulin-dependent diabetes mellitus), type II diabetes (non-insulin dependent diabetes mellitus), gestational diabetes, diabetes caused by administration of anti-psychotic agents, diabetes caused by administration of anti-depressant agents, diabetes caused by administration of steroid drugs, autoimmune diabetes, insulinopathies, diabetes due to pancreatic disease, diabetes associated with other endocrine diseases (such as Cushing's Syndrome, acromegaly, pheochromocytoma, glucagonoma, primary aldosteronism or somatostatinoma), type A insulin resistance syndrome, type B insulin resistance syndrome, lipatrophic diabetes, diabetes induced by β-cell toxins, and diabetes induced by drug therapy (such as diabetes induced by antipsychotic agents).

In one embodiment, the diabetes is type I diabetes.

In another embodiment, the diabetes is type II diabetes.

In another embodiment, the diabetes is gestational diabetes.

### Methods For Treating or Preventing a Diabetic Complication

The Compounds of Formula (I) are useful for treating or preventing a diabetic complication in a patient. Accordingly, in one embodiment, the present invention provides a method for treating a diabetic complication in a patient, comprising administering to the patient an effective amount of one or more Compounds of Formula (I).

Examples of diabetic complications treatable or preventable using the Compounds of Formula (I) include, but are not limted to, diabetic cataract, glaucoma, retinopathy, aneuropathy (such as diabetic neuropathy, polyneuropathy, mononeuropathy, autonomic neuropathy, microaluminuria and progressive diabetic neuropathyl), nephropathy, diabetic pain, gangrene of the feet, immune-complex vasculitis, systemic lupsus erythematosus (SLE), atherosclerotic coronary arterial disease, peripheral arterial disease, nonketotic hyperglycemic-hyperosmolar coma, foot ulcers, joint problems, a skin or mucous membrane complication (such as an infection, a shin spot, a candidal infection or necrobiosis lipoidica diabeticorumobesity), hyperlipidemia, hypertension, syndrome of insulin resistance, coronary artery disease, a fungal infection, a bacterial infection, and cardiomyopathy.

In one embodiment, the diabetic complication is neuropathy.

In another embodiment, the diabetic complication is retinopathy.

In another embodiment, the diabetic complication is nephropathy.

### Methods For Treating or Preventing Impaired Glucose Tolerance

The Compounds of Formula (I) are useful for treating or preventing impaired glucose tolerance in a patient.

Accordingly, in one embodiment, the present invention provides a method for treating impaired glucose tolerance in a patient, comprising administering to the patient an effective amount of one or more Compounds of Formula (I).

### Methods For Treating or Preventing Impaired Fasting Glucose

The Compounds of Formula (I) are useful for treating or preventing impaired fasting glucose in a patient.

Accordingly, in one embodiment, the present invention provides a method for treating impaired fasting glucose in a patient, comprising administering to the patient an effective amount of one or more Compounds of Formula (I).

### Combination Therapy

Accordingly, in one embodiment, the present invention provides methods for treating a Condition in a patient, the method comprising administering to the patient one or more Compounds of Formula (I), or a pharmaceutically acceptable salt or solvate thereof and at least one additional therapeutic agent that is not a Compound of Formula (I), wherein the amounts administered are together effective to treat or prevent a Condition.

When administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. The amounts of the various actives in such combination therapy may be different amounts (different dosage amounts) or same amounts (same dosage amounts).

In one embodiment, the one or more Compounds of Formula (I) is administered during at time when the additional therapeutic agent(s) exert their prophylactic or therapeutic effect, or vice versa.

In another embodiment, the one or more Compounds of Formula (I) and the additional therapeutic agent(s) are administered in doses commonly employed when such agents are used as monotherapy for treating a Condition.

In another embodiment, the one or more Compounds of Formula (I) and the additional therapeutic agent(s) are administered in doses lower than the doses commonly employed when such agents are used as monotherapy for treating a Condition.

In still another embodiment, the one or more Compounds of Formula (I) and the additional therapeutic agent(s) act synergistically and are administered in doses lower than the doses commonly employed when such agents are used as monotherapy for treating a Condition.

In one embodiment, the one or more Compounds of Formula (I) and the additional therapeutic agent(s) are present in the same composition. In one embodiment, this composition is suitable for oral administration. In another embodiment, this composition is suitable for intravenous administration.

The one or more Compounds of Formula (I) and the additional therapeutic agent(s) can act additively or synergistically. A synergistic combination may allow the use of lower dosages of one or more agents and/or less frequent administration of one or more agents of a combination therapy. A lower dosage or less frequent administration of one or more agents may lower toxicity of the therapy without reducing the efficacy of the therapy.

In one embodiment, the administration of one or more Compounds of Formula (I) and the additional therapeutic agent(s) may inhibit the resistance of a Condition to these agents.

In one embodiment, when the patient is treated for diabetes, a diabetic complication, impaired glucose tolerance or impaired fasting glucose, the other therapeutic is an antidiabetic agent which is not a Compound of Formula (I). In another embodiment, when the patient is treated for pain, the other therapeutic agent is an analgesic agent which is not a Compound of Formula (I).

In another embodiment, the other therapeutic agent is an agent useful for reducing any potential side effect of a Compound of Formula (I). Such potential side effects include, but are not limited to, nausea, vomiting, headache, fever, lethargy, muscle aches, diarrhea, general pain, and pain at an injection site.

In one embodiment, the other therapeutic agent is used at its known therapeutically effective dose. In another embodiment, the other therapeutic agent is used at its normally prescribed dosage. In another embodiment, the other therapeutic agent is used at less than its normally prescribed dosage or its known therapeutically effective dose.

Examples of antidiabetic agents useful in the present methods for treating diabetes or a diabetic complication include a sulfonylurea; an insulin sensitizer (such as a PPAR agonist, a DPP-IV inhibitor, a PTP-1B inhibitor and a glucokinase activator); a glucosidase inhibitor; an insulin secretagogue; a hepatic glucose output lowering agent; an anti-obesity agent; an antihypertensive agent; a meglitinide; an agent that slows or blocks the breakdown of starches and sugars in vivo; an histamine H₃ receptor antagonist; an antihypertensive agent, a sodium glucose uptake transporter 2 (SGLT-2) inhibitor; a peptide that increases insulin production; and insulin or any insulin-containing composition.

In one embodiment, the antidiabetic agent is an insulin sensitizer or a sulfonylurea.

Non-limiting examples of sulfonylureas include glipizide, tolbutamide, glyburide, glimepiride, chlorpropamide, acetohexamide, gliamilide, gliclazide, glibenclamide and tolazamide.

Non-limiting examples of insulin sensitizers include PPAR activators, such as troglitazone, rosiglitazone, pioglitazone and englitazone; biguanidines such as metformin and phenformin; DPP-IV inhibitors; PTP-1B inhibitors; and α-glucokinase activators, such as miglitol, acarbose, and voglibose.

Non-limiting examples of DPP-IV inhibitors useful in the present methods include sitagliptin, saxagliptin (Januvia™, Merck), denagliptin, vildagliptin (Galvus™, Novartis), alogliptin, alogliptin benzoate, ABT-279 and ABT-341 (Abbott), ALS-2-0426 (Alantos), ARI-2243 (Arisaph), B1-A and B1-B (Boehringer Ingelheim), SYR-322 (Takeda), MP-513 (Mitsubishi), DP-893 (Pfizer), RO-0730699 (Roche) or a combination of sitagliptin/metformin HCl (Janumet™, Merck).

Non-limiting examples of SGLT-2 inhibitors useful in the present methods include dapagliflozin and sergliflozin, AVE2268 (Sanofi-Aventis) and T-1095 (Tanabe Seiyaku).

Non-limiting examples of hepatic glucose output lowering agents include Glucophage and Glucophage XR.

Non-limiting examples of histamine H₃ receptor antagonist agents include the following compound:

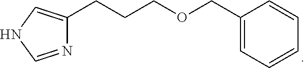

Non-limiting examples of insulin secretagogues include sulfonylurea and non-sulfonylurea drugs such as GLP-1, a GLP-1 mimetic, exendin, GIP, secretin, glipizide, chlorpropamide, nateglinide, meglitinide, glibenclamide, repaglinide and glimepiride.

Non-limiting examples of GLP-1 mimetics useful in the present methods include Byetta-Exanatide, Liraglutinide, CJC-1131 (ConjuChem, Exanatide-LAR (Amylin), BIM-51077 (Ipsen/LaRoche), ZP-10 (Zealand Pharmaceuticals), and compounds disclosed in International Publication No. WO 00/07617.

The term “insulin” as used herein, includes all formulations of insulin, including long acting and short acting forms of insulin.

Non-limiting examples of orally administrable insulin and insulin containing compositions include AL-401 from AutoImmune, and the compositions disclosed in U.S. Pat. Nos. 4,579,730; 4,849,405; 4,963,526; 5,642,868; 5,763,396; 5,824,638; 5,843,866; 6,153,632; 6,191,105; and International Publication No. WO 85/05029, each of which is incorporated herein by reference.

In one embodiment, the antidiabetic agent is an obesity agent.

Non-limiting examples of anti-obesity agents useful in the present methods for treating diabetes include a 5-HT2C agonist, such as lorcaserin; a neuropeptide Y antagonist; an MCR4 agonist; an MCH receptor antagonist; a protein hormone, such as leptin or adiponectin; an AMP kinase activator; and a lipase inhibitor, such as orlistat. Appetite suppressants are not considered to be within the scope of the anti-obesity agents useful in the present methods.

Non-limiting examples of antihypertensive agents useful in the present methods for treating diabetes include β-blockers and calcium channel blockers (for example diltiazem, verapamil, nifedipine, amlopidine, and mybefradil), ACE inhibitors (for example captopril, lisinopril, enalapril, spirapril, ceranopril, zefenopril, fosinopril, cilazapril, and quinapril), AT-1 receptor antagonists (for example losartan, irbesartan, and valsartan), renin inhibitors and endothelin receptor antagonists (for example sitaxsentan).

Non-limiting examples of meglitinides useful in the present methods for treating diabetes include repaglinide and nateglinide.

Non-limiting examples of insulin sensitizing agents include biguanides, such as metformin, metformin hydrochloride (such as GLUCOPHAGE® from Bristol-Myers Squibb), metformin hydrochloride with glyburide (such as GLUCOVANCE™ from Bristol-Myers Squibb) and buformin; glitazones; and thiazolidinediones, such as rosiglitazone, rosiglitazone maleate (AVANDIA™ from GlaxoSmithKline), pioglitazone, pioglitazone hydrochloride (ACTOS™, from Takeda) ciglitazone and MCC-555 (Mitstubishi Chemical Co.)

In one embodiment, the insulin sensitizer is a thiazolidinedione.

In another embodiment, the insulin sensitizer is a biguanide.

In another embodiment, the insulin sensitizer is a DPP-IV inhibitor.

In a further embodiment, the antidiabetic agent is a SGLT-2 inhibitor.

Non-limiting examples of antidiabetic agents that slow or block the breakdown of starches and sugars and are suitable for use in the compositions and methods of the present invention include alpha-glucosidase inhibitors and certain peptides for increasing insulin production. Alpha-glucosidase inhibitors help the body to lower blood sugar by delaying the digestion of ingested carbohydrates, thereby resulting in a smaller rise in blood glucose concentration following meals. Non-limiting examples of suitable alpha-glucosidase inhibitors include acarbose; miglitol; camiglibose; certain polyamines as disclosed in WO 01/47528 (incorporated herein by reference); voglibose. Non-limiting examples of suitable peptides for increasing insulin production including armlintide (CAS Reg. No. 122384-88-7 from Amylin; pramlintide, exendin, certain compounds having Glucagon-like peptide-1 (GLP-1) agonistic activity as disclosed in WO 00/07617 (incorporated herein by reference).

Non-limiting examples of orally administrable insulin and insulin containing compositions include AL-401 from AutoImmune, and the compositions disclosed in U.S. Pat. Nos. 4,579,730; 4,849,405; 4,963.526; 5,642.868; 5,763,396; 5,824,638; 5,843,866; 6,153,632; 6,191,105; and International Publication No. WO 85/05029, each of which is incorporated herein by reference.

Non-limiting examples of other analgesic agents useful in the present methods for treating pain include acetaminophen, an NSAID, an opiate or a tricyclic antidepressant.

In one embodiment, the other analgesic agent is acetaminophen or an NSAID.

In another embodiment, the other analgesic agent is an opiate.

In another embodiment, the other analgesic agent is a tricyclic antidepressant.

Non-limiting examples of NSAIDS useful in the present methods for treating pain include a salicylate, such as aspirin, amoxiprin, benorilate or diflunisal; an arylalkanoic acid, such as diclofenac, etodolac, indometacin, ketorolac, nabumetone, sulindac or tolmetin; a 2-arylpropionic acid (a “profen”), such as ibuprofen, carprofen, fenoprofen, flurbiprofen, loxoprofen, naproxen, tiaprofenic acid or suprofen; a fenamic acid, such as mefenamic acid or meclofenamic acid; a pyrazolidine derivative, such as phenylbutazone, azapropazone, metamizole or oxyphenbutazone; a coxib, such as celecoxib, etoricoxib, lumiracoxib or parecoxib; an oxicam, such as piroxicam, lornoxicam, meloxicam or tenoxicam; or a sulfonanilide, such as nimesulide.

Non-limiting examples of opiates useful in the present methods for treating pain include an anilidopiperidine, a phenylpiperidine, a diphenylpropylamine derivative, a benzomorphane derivative, an oripavine derivative and a morphinane derivative. Additional illustrative examples of opiates include morphine, diamorphine, heroin, buprenorphine, dipipanone, pethidine, dextromoramide, alfentanil, fentanyl, remifentanil, methadone, codeine, dihydrocodeine, tramadol, pentazocine, vicodin, oxycodone, hydrocodone, percocet, percodan, norco, dilaudid, darvocet or lorcet.

Non-limiting examples of tricyclic antidepressants useful in the present methods for treating pain include amitryptyline, carbamazepine, gabapentin or pregabalin.

The Compounds of Formula (I) can be combined with an H₁ receptor antagonist (i.e., the Compounds of Formula (I) can be combined with an H₁ receptor antagonist in a pharmaceutical composition, or the Compounds of Formula (I) can be administered with one or more H₁ receptor antagonists).

Numerous chemical substances are known to have histamine H₁ receptor antagonist activity and can therefore be used in the methods of this invention. Many H₁ receptor antagonists useful in the methods of this invention can be classified as ethanolamines, ethylenediamines, alkylamines, phenothiazines or piperidines. Representative H₁ receptor antagonists include, without limitation: astemizole, azatadine, azelastine, acrivastine, brompheniramine, cetirizine, chlorpheniramine, clemastine, cyclizine, carebastine, cyproheptadine, carbinoxamine, descarboethoxyloratadine, diphenhydramine, doxylamine, dimethindene, ebastine, epinastine, efletirizine, fexofenadine, hydroxyzine, ketotifen, loratadine, levocabastine, meclizine, mizolastine, mequitazine, mianserin, noberastine, norastemizole, picumast, pyrilamine, promethazine, terfenadine, tripelennamine, temelastine, trimeprazine and triprolidine. Other compounds can readily be evaluated to determine activity at H₁ receptors by known methods, including specific blockade of the contractile response to histamine of isolated guinea pig ileum. See for example, WO98/06394 published Feb. 19, 1998.

Those skilled in the art will appreciate that the H₁ receptor antagonist is used at its known therapeutically effective dose, or the H₁ receptor antagonist is used at its normally prescribed dosage.

Preferably, said H₁ receptor antagonist is selected from: astemizole, azatadine, azelastine, acrivastine, brompheniramine, cetirizine, chlorpheniramine, clemastine, cyclizine, carebastine, cyproheptadine, carbinoxamine, descarboethoxyloratadine, diphenhydramine, doxylamine, dimethindene, ebastine, epinastine, efletirizine, fexofenadine, hydroxyzine, ketotifen, loratadine, levocabastine, meclizine, mizolastine, mequitazine, mianserin, noberastine, norastemizole, picumast, pyrilamine, promethazine, terfenadine, tripelennamine, temelastine, trimeprazine or triprolidine.

More preferably, said H₁ receptor antagonist is selected from: astemizole, azatadine, azelastine, brompheniramine, cetirizine, chlorpheniramine, clemastine, carebastine, descarboethoxyloratadine, diphenhydramine, doxylamine, ebastine, fexofenadine, loratadine, levocabastine, mizolastine, norastemizole, or terfenadine.

Most preferably, said H₁ receptor antagonist is selected from: azatadine, brompheniramine, cetirizine, chlorpheniramine, carebastine, descarboethoxy-loratadine, diphenhydramine, ebastine, fexofenadine, loratadine, or norastemizote.

Even more preferably, said H₁ antagonist is selected from loratadine, descarboethoxyloratadine, fexofenadine or cetirizine. Still even more preferably, said H₁ antagonist is loratadine or descarboethoxyloratadine.

In one preferred embodiment, said H₁ receptor antagonist is loratadine.

In another preferred embodiment, said H₁ receptor antagonist is descarboethoxyloratadine.

In still another preferred embodiment, said H₁ receptor antagonist is fexofenadine.

In yet another preferred embodiment, said H₁ receptor antagonist is cetirizine.

Preferably, in the above methods, allergy-induced airway responses are treated.

Also, preferably, in the above methods, allergy is treated.

Also, preferably, in the above methods, nasal congestion is treated.

In the methods of this invention wherein a combination of an H₃ antagonist of this invention (compound of formula I) is administered with a H₁ antagonist, the antagonists can be administered simultaneously or sequentially (first one and then the other over a period of time). In general, when the antagonists are administered sequentially, the H₃ antagonist of this invention (compound of formula I) is administered first.

The doses and dosage regimen of the other agents used in the combination therapies of the present invention for the treatment or prevention of a Condition can be determined by the attending clinician, taking into consideration the approved doses and dosage regimen in the package insert; the age, sex and general health of the patient; and the type and severity of the viral infection or related disease or disorder. When administered in combination, the Compound(s) of Formula (I) and the other agent(s) for treating diseases or conditions listed above can be administered simultaneously or sequentially. This is particularly useful when the components of the combination are given on different dosing schedules, e.g., one component is administered once daily and another every six hours, or when the preferred pharmaceutical compositions are different, e.g., one is a tablet and one is a capsule. A kit comprising the separate dosage forms is therefore advantageous.

Generally, a total daily dosage of the one or more Compounds of Formula (I) and the additional therapeutic agent(s) can, when administered as combination therapy, range from about 0.1 to about 2000 mg per day, although variations will necessarily occur depending on the target of the therapy, the patient and the route of administration. In one embodiment, the dosage is from about 0.2 to about 100 mg/day, administered in a single dose or in 2-4 divided doses. In another embodiment, the dosage is from about 1 to about 500 mg/day, administered in a single dose or in 2-4 divided doses. In another embodiment, the dosage is from about 1 to about 200 mg/day, administered in a single dose or in 2-4 divided doses. In still another embodiment, the dosage is from about 1 to about 100 mg/day, administered in a single dose or in 2-4 divided doses. In yet another embodiment, the dosage is from about 1 to about 50 mg/day, administered in a single dose or in 2-4 divided doses. In a further embodiment, the dosage is from about 1 to about 20 mg/day, administered in a single dose or in 2-4 divided doses.

### Compositions and Administration

In one embodiment, the invention provides compositions comprising an effective amount of one or more Compounds of Formula (I) or a pharmaceutically acceptable salt, solvate, ester or prodrug thereof, and a pharmaceutically acceptable carrier.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Pa.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g., nitrogen.

Also included are solid form preparations which are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

In one embodiment, the Compound of Formula (I) is administered orally.

In another embodiment, the Compound of Formula (I) is administered parenterally.

In another embodiment, the Compound of Formula (I) is administered intravenously.

In one embodiment, the pharmaceutical preparation is in a unit dosage form. In such form, the preparation is subdivided into suitably sized unit doses containing appropriate quantities of the active component, e.g., an effective amount to achieve the desired purpose.

The quantity of active compound in a unit dose of preparation is from about 0.1 to about 2000 mg. Variations will necessarily occur depending on the target of the therapy, the patient and the route of administration. In one embodiment, the unit dose dosage is from about 0.2 to about 1000 mg. In another embodiment, the unit dose dosage is from about Ito about 500 mg. In another embodiment, the unit dose dosage is from about 1 to about 100 mg/day. In still another embodiment, the unit dose dosage is from about 1 to about 50 mg. In yet another embodiment, the unit dose dosage is from about 1 to about 10 mg.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 1 mg/day to about 300 mg/day, preferably 1 mg/day to 75 mg/day, in two to four divided doses.

When the invention comprises a combination of at least one Compound of Formula (I) and an additional therapeutic agent, the two active components may be co-administered simultaneously or sequentially, or a single pharmaceutical composition comprising at least one Compound of Formula (I) and an additional therapeutic agent in a pharmaceutically acceptable carrier can be administered. The components of the combination can be administered individually or together in any conventional dosage form such as capsule, tablet, powder, cachet, suspension, solution, suppository, nasal spray, etc. The dosage of the additional therapeutic agent can be determined from published material, and may range from about 1 to about 1000 mg per dose. In one embodiment, when used in combination, the dosage levels of the individual components are lower than the recommended individual dosages because of the advantageous effect of the combination.

In one embodiment, the components of a combination therapy regime are to be administered simultaneously, they can be administered in a single composition with a pharmaceutically acceptable carrier.

In another embodiment, when the components of a combination therapy regime are to be administered separately or sequentially, they can be administered in separate compositions, each containing a pharmaceutically acceptable carrier.

The components of the combination therapy can be administered individually or together in any conventional dosage form such as capsule, tablet, powder, cachet, suspension, solution, suppository, nasal spray, etc.

### Kits

In one aspect, the present invention provides a kit comprising a effective amount of one or more Compounds of Formula (I), or a pharmaceutically acceptable salt or solvate of the compound and a pharmaceutically acceptable carrier, vehicle or diluent.

In another aspect the present invention provides a kit comprising an amount of one or more Compounds of Formula (I), or a pharmaceutically acceptable salt or solvate of the compound and an amount of at least one additional therapeutic agent listed above, wherein the combined amounts are effective for treating or preventing a Condition in a patient.

When the components of a combination therapy regime are to are to be administered in more than one composition, they can be provided in a kit comprising in a single package, one container comprising a Compound of Formula (I) in pharmaceutically acceptable carrier, and one or more separate containers, each comprising one or more additional therapeutic agents in a pharmaceutically acceptable carrier, with the active components of each composition being present in amounts such that the combination is therapeutically effective.

The present invention is not to be limited by the specific embodiments disclosed in the examples that are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

A number of references have been cited herein, the entire disclosures of which are incorporated herein by reference.

1-48. (canceled)

49. A compound of the formula I: 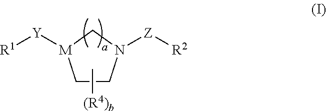
wherein:
R¹ is: 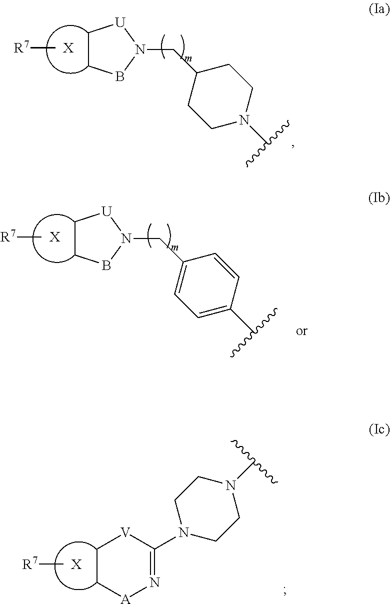
R² is alkyl, alkenyl, aryl, cycloalkyl, heterocycloalkyl or heteroaryl, any of which can be optionally substituted with R³;
R³ represents from 1 to 3 substituents, each independently selected from H, halo, alkyl, —OH, —O-alkyl, hydroxyalkyl, aryl, —O-aryl, haloalkyl, —NO₂, —C(O)OR⁹, —N(R⁹)₂, —C(O)N(R⁹)₂, -alkylene-N(R⁹)₂, —NHC(O)R⁹, —NHC(O)OR⁹, —NHS(O)₂R⁹, —S(O)₂N(R⁹)₂ and —CN;
R⁴ is halo, alkyl, —OH, —O-alkyl, haloalkyl or —CN;
each occurrence of R⁵ is independently H, alkyl, haloalkyl, aryl, heteroaryl, cycloalkyl or heterocycloalkyl;
R⁶ is alkyl, aryl or heteroaryl;
each occurrence of R⁷ is independently hydrogen, halo, —OH, alkyl, —O-alkyl, haloalkyl, —O-haloalkyl, —NO₂, —C(O)R⁵, —N(R⁵)₂, —C(O)N(R⁵)₂, —NHC(O)R⁵, —NHS(O)₂R⁵, —S(O)₂N(R⁵)₂ or —CN;
each occurrence of R⁸ is independently H or alkyl;
each occurrence of R⁹ is independently H, alkyl, haloalkyl, aryl, heteroaryl, cycloalkyl or heterocycloalkyl;
each occurrence of R¹⁰ is independently hydrogen, halogen, —OH, alkyl, —O-alkyl, haloalkyl, —O-haloalkyl, —NO₂, —C(O)OR⁶, —N(R⁵)₂, —C(O)N(R⁵)₂, —NHC(O)R⁶, —NHS(O)₂R⁶, —S(O)₂N(R⁵)₂, —C(O)R⁵ or —CN;
R¹¹ is hydrogen, alkyl, —C(O)R⁶ or —S(O)₂R⁶;
R¹² is hydrogen, alkyl, haloalkyl or —C(O)R⁵;
R¹³ is hydrogen, alkyl or haloalkyl;
R¹⁴ represents 1 to 3 substituents, each independently selected from the group consisting of H, cycloalkyl, heterocycloalkyl, aryl, heteroaryl, haloalkyl, halo, —CN, —OH, —O-alkyl, —O-haloalkyl, —NO₂, and —N(R⁸)₂;
R¹⁵ represents 1 to 3 substituents, each independently selected from cycloalkyl, heterocycloalkyl, aryl, heteroaryl and haloalkyl;
A is a bond, —O—, —S—, —O-alkylene-, —S-alkylene-, —C(R¹⁰)ₚC(R¹²)— or) —N(R¹²)—C(R¹⁰)ₚC(R¹²)—;
B is a bond, —O—, —S—, —O-alkylene-, —S-alkylene-, —C(R¹⁰)ₚC(R¹²)— or —N(R¹²)—C(R¹⁰)ₚC(R¹²)—, such that when R¹ is (Ia) or (Ib) and B is —O— or —S—, then U is other than —O— or —S—;
M is —CH—, —C(halo)- or —N—;
Q is —O—, —S—, —S(O)— or —S(O)₂—;
U is a bond, —O—, —S—, —O-alkylene-, —S-alkylene-, —C(R¹⁰)ₙC(R¹²)—, —N(R¹¹)C(R¹²)C(R¹²)—, —N(R¹¹)C(R¹²)C(R¹⁰)C(R¹²)—, -QC(R¹³)C(R¹⁰)C(R¹²)—, —C(R¹²)N(R¹¹)C(R¹²)C(R¹²)—, -QC(R¹³)C(R¹²)—, or —C(R¹³)QC(R¹³)C(R¹²)—, such that when R¹ is (Ia) or (Ib) and B is —O— or —S—, then U is other than —O— or —S—;
V is a bond, —O—, —S—, —O-alkylene-, —S-alkylene-, —C(R¹⁰)ₚC(R¹²)—, —C(R¹²)N(R¹¹)C(R¹²)—, —C(R¹³)QC(R¹³)—, —N(R¹¹)C(R¹²)C(R¹⁰)—, -QC(R¹³)C(R¹⁰)—, —N(R¹¹)C(R¹²)— or -QC(R¹³)—;
X is aryl, or heteroaryl, each of which can be optionally fused to a benzene ring;
Y is —C(O)—, —S—, —S(O)—, —S(O)₂—, —CH₂— or —O—, such that if Y is —O— or —S—, then M is other than N and R¹ is (Ib);
Z is a bond, alkylene, -alkylene-cycloalkylene-alkylene-, -alkylene-heterocycloalkylene-alkylene-, —CH(R⁸)—CH(R⁸)—O—, —CH(R⁸)—CH(R⁸)—N—, —CH(R⁸)—(C₁-C₅ alkylene)-R¹⁴, —CH(R⁸)—C(R⁸)═C(R⁸)—, —CH(R⁸)—C(R⁸)═C(R⁸)—(C₁-C₃ alkylene)-R¹⁴, wherein any alkylene moiety of a Z group can be optionally substituted with one or more R¹⁵ groups, such that when Z is an -alkylene-heterocycloalkylene-alkylene-group substituted with one or more R¹⁵ groups and the heterocycloalkylene group is bonded through a ring nitrogen atom, then Z is —(C₂-C₄ alkylene)-heterocycloalkylene-alkylene-;
a is 1, 2, or 3;
b is 0, 1, or 2;
m is 0, 1 or 2; and
each occurrence of p is 0, 1, 2 or 3;
or a pharmaceutically acceptable salt thereof.

50. The compound of claim 49, wherein R¹ is:

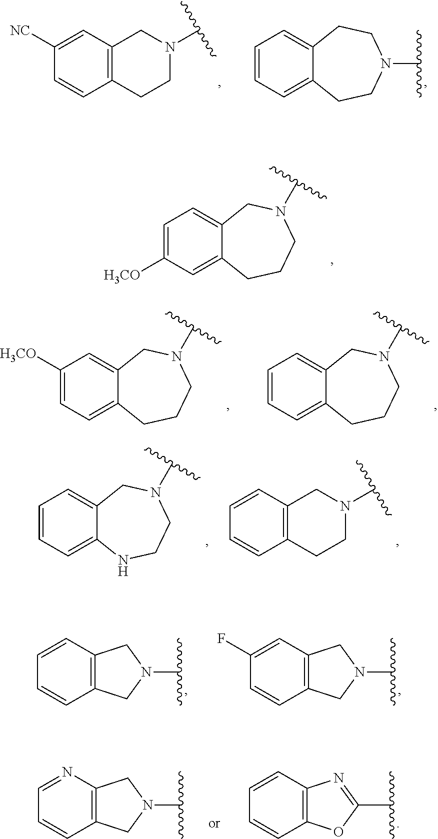

51. The compound of claim 49, wherein R² is:

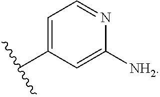

52. The compound of claim 49, wherein m is 0, m is 1 and a is 2.

53. The compound of claim 49, wherein M is —CH— or —C(halo)-.

54. The compound of claim 49, wherein Y is —C(O)— or —O—.

55. The compound of claim 49, wherein Z is —CH₂— or —CH(CH₃)—.

56. The compound of claim 49, wherein A is methylene or propylene.

57. The compound of claim 49, wherein A is —CH₂—CH₂—NH—, where the —NH— is attached to ring X.

58. The compound of claim 49, wherein a is 2, M is —CH— or —CF—, Y is —C(O)—, Z is alkylene, R² is heteroaryl and R¹ is:

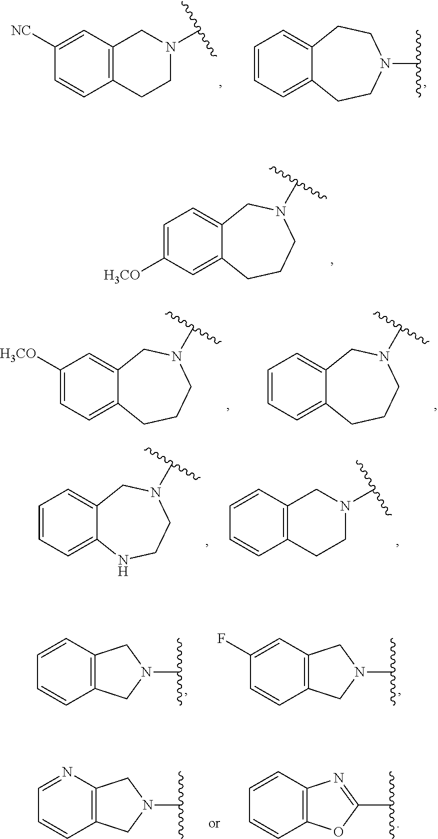

59. A compound which is selected from the group consisting of: 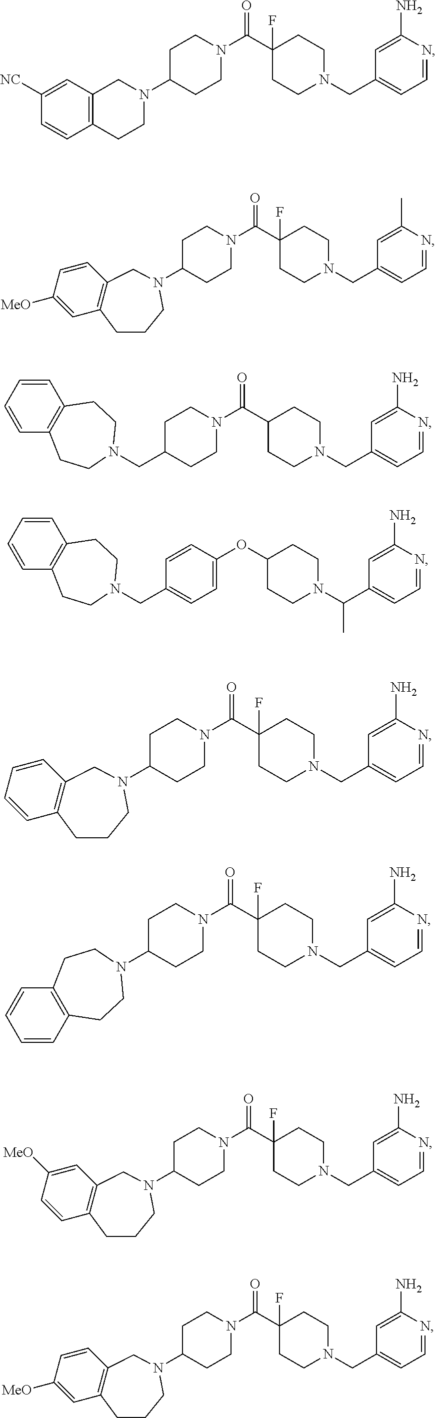 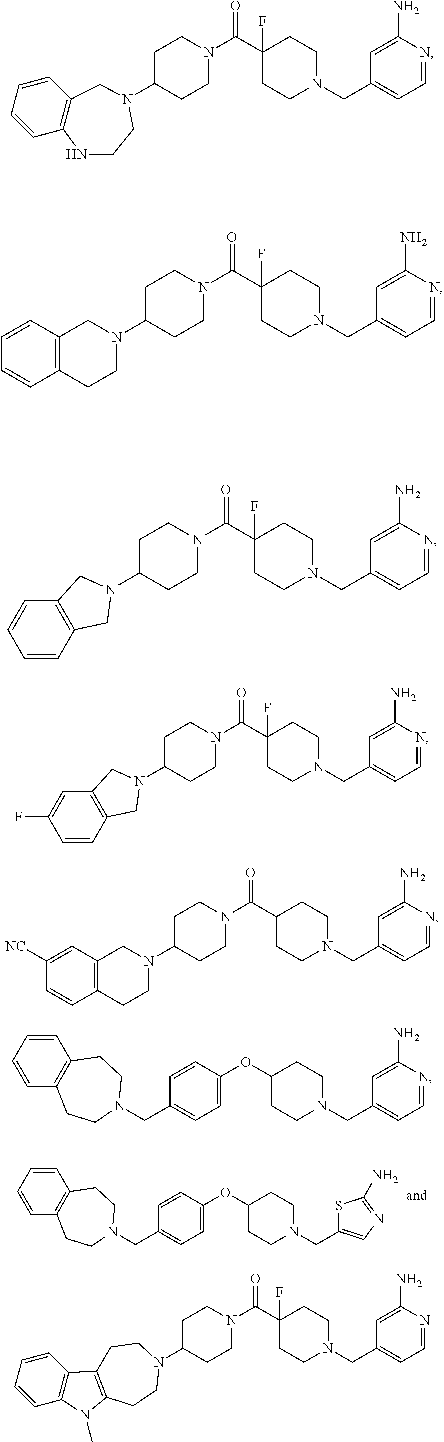
or a pharmaceutically acceptable salt thereof.

60. A pharmaceutical composition comprising the compound of claim 49 or a pharmaceutically acceptable salt thereof and a pharmaceutically acceptable carrier.

61. A method of treating allergy, an allergy-induced airway response, congestion, a cardiovascular disease, an inflammatory disease, a gastrointestinal disorder, a neurological disorder, a metabolic disorder, obesity or an obesity-related disorder, diabetes, a diabetic complication, impaired glucose tolerance or impaired fasting glucose in a patient, comprising administering to the patient an effective amount of the compound of claim 49 or a pharmaceutically acceptable salt thereof.
